(19) United States
(12) Patent Application Publication
(10) Pub. No.: US 2004/0053962 A1
(43) Pub. Date: Mar. 18, 2004

(54) Methods for inhibiting proliferation and inducing apoptosis in cancer cells

(76) Inventor: Thomas E. Adrian, Chicago, IL (US)

Correspondence Address: Peter Rogalskyj, Rogalskyj & Weyand, P O Box 44, Livonia, NY, 14487-0044, US

(21) Appl. No.: 10/275,590

(22) PCT Filed: May 8, 2001
(86) PCT No.: PCT/US01/40697

(51) Int. Cl. A61K 31/47; A61K 31/095; A61K 31/075

(52) U.S. Cl. 514/311; 514/706; 514/718

(57) ABSTRACT

Disclosed are methods of decreasing proliferation of adenocarcinoma cancer cells, or of inducing apoptosis of adenocarcinoma cancer cells, or of inducing differentiation of adenocarcinoma cancer cells into non-cancerous cells. One such method includes contacting the adenocarcinoma cancer cells with a compound under conditions effective for the compound to inhibit binding of leukotriene B4 to leukotriene B4 receptor. In another such method, the method includes contacting the adenocarcinoma cancer cells with 2-(2-propyl-3-(3-(2-ethyl-4-(4-fluorophenyl)-5-hydroxyphenoxy)propoxy)phenoxy)benzoic acid or a pharmaceutically acceptable salt, solvate, or congener thereof. Also disclosed are methods of treating adenocarcinomas in a subject. One method includes administering to the subject an amount of a compound effective to inhibit binding of leukotriene B4 to leukotriene B4 receptor. Another method includes administering 2-(2-propyl-3-(3-(2-ethyl-4-(4-fluoropheyl)-5-hydroxyphenoxy)propoxy)phenoxy)benzoic acid or a pharmaceutically acceptable salt, solvate, or congener thereof, to the subject.

The present invention was made with the support of the National Cancer Institute Contract No. P50 CA72712. The Federal Government may have certain rights in this invention.

### FIELD OF THE INVENTION

The subject invention is directed generally to methods for decreasing proliferation of cancer cells, or of inducing apoptosis of cancer cells, or of inducing differentiation of cancer cells into non-cancerous cells and to methods for treating adenocarcinoma in a subject.

### BACKGROUND OF THE INVENTION

Pancreatic cancer is one of the most enigmatic and aggressive malignant diseases facing oncologists (Parker et al., “Cancer Statistics. 1996,” CA Cancer J. Clin., 46:5-27 (1996) (“Parker”)). It is now the fourth leading cause of cancer death in both men and women in the United States, and the incidence of this disease has significantly increased over the past 20 years (Parker; Trede et al., “Survival After Pancreaticoduodenectomy: 118 Consecutive Resections Without an Operative Mortality,” Ann. Surg., 211:447-458 (1990); Cameron et al., “One Hundred and Forty-five Consecutive Pancreatico-duodenectomies Without Mortality,” Ann. Surg.,217:430-438 (1993); Horward, “Pancreatic Adenocarcinoma,” Curr. Prob. in Cancer, 20:286-293 (1996) (“Horward”); Poston et al., Gut. Biology of Pancreatic Cancer, 32:800-812 (1991) (“Poston”); and Black et al., “Treatment of Pancreatic Cancer: Current Limitations, Future Possibilities,” Oncology, 10:301-307 (1996) (“Black”)). Pancreatic cancer is responsible for 27,000 deaths per year in the United States. Because of lack of early diagnosis and poor therapeutic responsiveness of pancreatic cancer, less than 2% of patients survive beyond five years, and the median expectation of life after diagnosis of pancreatic cancer is less than 6 months (Horward; Poston; and Black).

Colonic cancer is the second most common form of cancer in the United States (Doll et al., “Mortality in Relation to Smoking: 20 Years' Observations on Male British Doctors,” BMJ, 2:1525-1536 (1976); Hruban et al., “Molecular Diagnosis of Cancer and Micrometastases,” Adv. Anat. Pathol., 5:175-178 (1998) (“Hruban”); Figueredo et al., “Adjuvant Therapy for Stage II Colon Cancer After Complete Resection. Provincial Gastrointestinal Disease Site Group,” Cancer Prev. Control, 1:379-92 (1997) (“Figueredo”); Ness et al., “Outcome States of Colorectal Cancer: Identification and Description Using Patient Focus Groups,” Am. J. Gastroenterol., 93:1491-7 (1998) (“Ness”); Trehu et al., “Cost of Screening for Colorectal Cancer: Results of a Community Mass Screening Program and Review of Literature,” South Med. J., 85:248-253 (1992); and Wingo et al., “Cancer Statistics,” CA Cancer J. Clin., 45:8-30 (1995) (“Wingo”)). Colonic cancer occurs in more than 138,000 patients and is responsible for more than 55,000 deaths in the United States each year (Wingo). Up to 70% of patients with colonic cancer develop hepatic metastasises by the time of death, indicating that non-detectable micro-metastases are present at the time of surgery (Hruban; Figueredo; and Ness). Furthermore, metastatic cancer is often not responsive to standard chemotherapeutic regimens, resulting in treatment failure (Figueredo and Ness). The overall response of advanced or non-resectable colorectal cancer patients to chemotherapeutic agents varies from 26 to 44 percent. For example, less than one third of colorectal cancer patients with liver metastases respond to treatment with agents such as 5-FU and leucovorin (Id.).

Breast cancer has the highest incidence of any cancer in women with the diagnosis being made in more than 275,000 per year in the USA (Richards et al., “Influence of Delay on Survival in Patients with Breast Cancer: A Systematic Review,” Lancet, 353:1119-26 (1999); Norton, “Adjuvant Breast Cancer Therapy: Current Status and Future Strategies—Growth Kinetics and the Improved Drug Therapy of Breast Cancer,” Semin. Oncol., 26:1-4 (1999); Morrow et al., “Current Controversies in Breast Cancer Management,” Curr. Probl. Surg., 36:163-216 (1999); and Ruppert et al., “Gene Therapy Strategies for Carcinoma of the Breast,” Breast Cancer Res. Treatment, 44:93-114 (1997)). Even though five year survival has increased to more than 80%, more than 77,000 women still die from this disease each year (Id.).

Thus, another dimension in chemotherapeutic agents for pancreatic, colonic, and breast cancer would be extremely beneficial, especially to control metastatic and unresectable disease.

### SUMMARY OF THE INVENTION

The present invention relates to a method of decreasing proliferation of adenocarcinoma cancer cells, or of inducing apoptosis of adenocarcinoma cancer cells, or of inducing differentiation of adenocarcinoma cancer cells into non-cancerous cells. A sample which includes adenocarcinoma cancer cells is contacted with a compound under conditions effective to inhibit binding of leukotriene B4 to leukotriene B4 receptor.

The present invention also relates to a method of treating adenocarcinoma in a subject. The method includes administering to the subject an amount of a compound effective to inhibit binding of leukotriene B4 to leukotriene B4 receptor.

The present invention also relates to a method of decreasing proliferation of adenocarcinoma cancer cells, or of inducing apoptosis of adenocarcinoma cancer cells, or of inducing differentiation of adenocarcinoma cancer cells into non-cancerous cells. The method includes contacting a sample which contains adenocarcinoma cancer cells with a compound having the formula (“Formula I”):

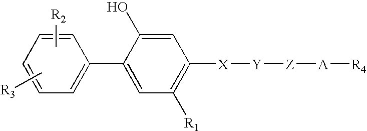

or a pharmaceutically acceptable salt or solvate thereof, wherein:

R₁ is C₁-C₅ alkyl, C₂-C₅ alkenyl, C₂-C₅ alkynyl, C₁-C₄ alkoxy, (C₃-C₄ alkyl)thio, halo, or R₂-substitutedphenyl;

each of R₂ and R₃ is independently hydrogen, halo, hydroxy, C₁-C₄ alkyl, C₁-C₄ alkoxy, (C₁-C₄ alkyl)-S(O)_{q}—, trifluoromethyl, or di-(C₁-C₃ alkyl) amino;

X is —O—, —S—, —C (═O), or —CH₂—, and Y is —O— or —CH₂—; or, when taken together, —X—Y— is —CH═CH— or —C≡C—;

Z is a straight or branched chain C₁-C₁₀ alkylidenyl;

A is a bond, —O—, —S—, —CH═CH—, or —CRₐR_{b}—, where each of Rₐ and R_{b} is independently hydrogen, C₁-C₅ alkyl, or R₇-substituted phenyl, or Rₐ and R_{b}, when taken together with the carbon atom to which they are attached, form a C₄-C₈ cycloalkyl ring;

R₄ is a R₆ or R₄ is a moiety having one of the following formulae:

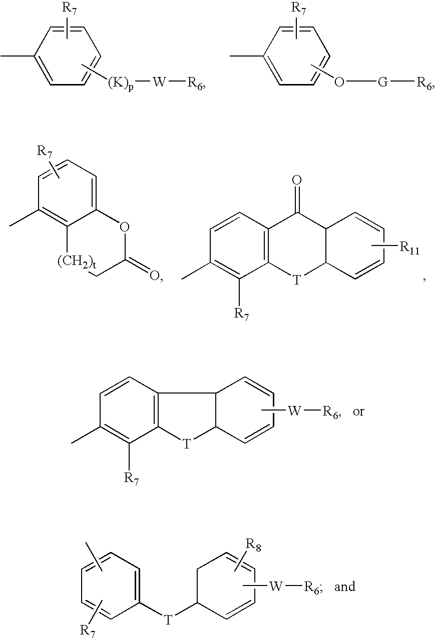

wherein:

each R₆ is independently —COOH, 5-tetrazolyl, —CON(R₉)₂, or —CONHSO₂R₁₀;

each R₇ is hydrogen, C₁-C₄ alkyl, C₂-C₅ alkenyl, C₂-C₅ alkynyl, benzyl, methoxy, —W—R₆, -T-G-R₆, (C₁-C₄ alkyl)-T-(C₁-C₄ alkylidenyl)-O—, or hydroxy;

R₈ is hydrogen or halo;

each R₉ is independently hydrogen, phenyl, or C₁-C₄ alkyl, or R₉, when taken together with the nitrogen atom to which they are attached, form a morpholino, piperidino, piperazino, or pyrrolidino group;

R₁₀ is C₁-C₄ alkyl or phenyl;

R₁₁ is R₂, —W—R₆, or -T-G-R₆;

each W is a bond or straight or branched chain divalent hydrocarbyl radical of one to eight carbon atoms;

each G is a straight or branched chain divalent hydrocarbyl radical of one to eight carbon atoms;

each T is a bond, —CH₂—, —O—, —NH—, —NHCO—, —C(═O)—, or —S(O)_{q}—;

K is —C(═O)— or —CH(OH)—;

each q is independently 0, 1, or 2;

p is 0 or 1; and

t is 0 or 1;

provided that, when X is —O— or —S—, Y is not —O—;

further provided that, when A is —O— or —S—, R₄ is not R₆;

further provided that, when A is —O— or —S— and Z is a bond, Y is not —O—; and

further provided that W is not a bond when p is 0.

The present invention also relates to a method of treating adenocarcinoma in a subject. The method includes administering, to the subject, an therapeutically effective amount of a compound of Formula I, or a pharmaceutically acceptable salt or solvate thereof.

### BRIEF DESCRIPTION OF THE DRAWINGS

FIGS. 1A and 1B are bar graphs showing the concentration-dependent inhibition (FIG. 1A) and time-course effects (FIG. 1B) of the LTB4 receptor antagonist, LY293111, on thymidine incorporation in a MiaPaCa-2 pancreatic cancer cell line. Results are expressed as % of control; *=P<0.05, **=P<0.01, ***=P<0.001 compared with control; and the data represent results from four separate experiments.

FIG. 2 is a graph showing the time-course effect of LY293111 on cell number in MiaPaCa-2 pancreatic cancer cells. Cells were treated with 500 nM LY293111 for 24, 48, and 72 hours, and the results (solid line, ▪) are expressed as relative cell numbers/well. Control data (dashed line, ▴) are also shown. *=P<0.05; **=P<0.01; and ***=P<0.001 compared with control. The data represent results from four separate experiments.

FIGS. 3A-3D are phase contrast micrograph images. FIG. 3B shows the morphological changes induced by 250 nM LY293111 in MiaPaCa-2 pancreatic cancer cells after 12 hours relative to control MiaPaCa-2 pancreatic cancer cells (FIG. 3A). FIG. 3D shows the morphological changes induced by 250 nM LY293111 in LoVo colonic cancer cells relative to control LoVo colonic cancer cells (FIG. 3D).

FIGS. 4A and 4B are bar graphs showing the effects of different concentrations of the LTD4 antagonist, LY171883 on thymidine incorporation in pancreatic cancer cell lines MiaPaCa-2 (FIG. 4A) and HPAF (FIG. 4B) after 48 hours. Results are expressed as % of control; *=P<0.05, **=P<0.01, ***=P<0.001 compared with control; and the data represent results from four separate experiments.

FIG. 5A is a bar graph showing the effects of different concentrations of LTB4 on thymidine incorporation in MiaPaCa-2 cells after 24 hours. FIGS. 5B and 5C are bar graphs showing the effects of 100 nM of LTB4 on thymidine incorporation in MiaPaCa-2 cells (FIG. 5B) and HPAF cells (FIG. 5C) for 24, 48, and 72 hours. Results of thymidine incorporation are expressed as cpm; *=P<0.05, **=P<0.01, ***=P<0.001 compared with control; and the data represent results from four separate experiments.

FIGS. 6A and 6B are bar graphs showing the effect of 250 nM LY293111 on the stimulatory effects of 100 nM LTB4 on pancreatic cancer cells MiaPaCa-2 (FIG. 6A) and HPAF (FIG. 6B) after 24 hours. Results are expressed as % of control; *=P<0.05, **=P<0.01, ***=P<0.001 compared with control; and the data represent results from four separate experiments.

FIG. 7 is an image of an agarose gel electrophoresis showing DNA fragmentation reflecting LY293111-induced apoptosis in MiaPaCa-2 cells. Cells were treated with 250, 500, and 1000 nM LY293111 for 48 hours. Total cellular DNA was separated on a 1.8% Agarose visualized by ethidium bromide staining. The results are representative of three separate experiments.

FIGS. 8A-8C are dot plots showing TUNEL assay results of MiaPaCa-2 pancreatic cancer cells treated with 250 nM (FIG. 8B) and 500 nM (FIG. 8C) LY293111 for 48 hours, as compared to control (FIG. 8A). The increase of fluorescence events in the upper right quadrant is due to UTP labeling of fragmented DNA, and the results are representative of three separate experiments.

FIGS. 9A-9F are bar graphs showing the effects of LY293111 on proliferation of pancreatic and colonic cancer cell lines. FIGS. 9A, 9C, and 9D show the dosage effects of LY293111 on pancreatic cancer cell lines MiaPaCa2, HPAF, and Capan2, respectively. FIG. 9B shows the time dependent effects of LY293111 on pancreatic cancer cell line MiaPaCa2. FIGS. 9E and 9F show the effects of 0.5 μM LY293111 on cell number as a function of time for pancreatic cancer cell line MiaPaCa2 (FIG. 9E) and for colonic cancer cell line LoVo (FIG. 9F).

FIGS. 10A and 10B are images of western blots showing the effects of LTB4 and LY293111, respectively, on Bcl-2 expression in MCF-7 breast cancer cells.

FIG. 11 is a graph of tumor volume in athymic mice xenografted with human pancreatic cell line AsPc-1 as a function of treatment time with oral LY293111 (▴) vs control (●)

### DETAILED DESCRIPTION OF THE INVENTION

The present invention relates to a method of decreasing proliferation of adenocarcinoma cancer cells, or of inducing apoptosis of adenocarcinoma cancer cells, or of inducing differentiation of adenocarcinoma cancer cells into non-cancerous cells. The method includes contacting a sample which includes adenocarcinoma cancer cells with a compound which inhibits binding of leukotriene B4 to leukotriene B4 receptor. “Inhibit” and its other forms (e.g., “inhibiting”) are meant include any degree of inhibition (e.g., more than about 5%, more than about 10%, more than about 20%, more than about 30%, more than about 40%, more than about 50%, more than about 60%, more than about 70%, more than about 80%, more than about 90%, more than about 95%, more than about 98%, more than about 99%) up to and including complete prevention (100% inhibition).

The mechanism by which binding of leukotriene B4 to leukotriene B4 receptor is inhibited is not particularly critical to the practice of the present invention. The inhibition can be direct, as in the case where the compound interferes directly with the binding of leukotriene B4 to leukotriene B4 receptor (e.g., by binding directly to leukotriene B4 receptor, as explained in more detail below), or it can be indirect, as in the case where the compound interferes with either the production of leukotriene B4 and/or leukotriene B4 receptor, as further explained below.

For example, the compound can inhibit binding of leukotriene B4 to leukotriene B4 receptor by binding to leukotriene B4 receptor. Preferably, the binding of the compound to leukotriene B4 receptor is specific, i.e., the compound does not bind substantially to other biological materials that are present in the sample. For example, it is preferred that the IC₅₀ of the compound to leukotriene B4 receptor be greater than about 1.1 times, more preferably greater than about 1.5 times, still more preferably greater than about 2 times, still more preferably greater than about 5 times, still more preferably greater than about 10 times the IC₅₀ of the compound to all other biological materials in the sample. The degree of binding of the compound to leukotriene B4 receptor is preferably greater (i.e., has a higher IC₅₀) than the binding of leukotriene B4 to leukotriene B4 receptor, although the desired effect can be achieved in some circumstances even when the degree of binding of the compound to leukotriene B4 receptor is less (i.e., has a lower IC₅₀) than the binding of leukotriene B4 to leukotriene B4 receptor.

“Binding”, as used herein, is meant to include irreversible binding and reversible binding. The binding can be the result of thermodynamic forces (e.g., favorable equilibrium constants) or kinetic considerations (e.g., favorable on/off rate constants), and it can be the result of chemical interactions (e.g., covalent bonding, ionic bonding, hydrogen bonding, van der Waals bonding, chelation, pi-sigma bonding, and/or pi-pi bonding) or physical interactions (e.g., surface phenomena, entrapment, etc.). As used in the context of “binding” of the compound with a binding partner (e.g., binding of the compound to a leukotriene B4 receptor), “binding” is also meant to include any interaction of the compound with its binding partner which results in a decrease in the ability of the binding partner to fulfill its physiological role. For example, in the case where the binding partner is leukotriene B4 receptor, “binding” of the compound to leukotriene B4 receptor is meant to include any interaction or combination of interactions between the compound and leucotriene B4 receptor which result in physical, chemical, and/or other changes in leucotriene B4 receptor which, in turn, decreases the ability of leucotriene B4 receptor to bind to leucotriene B4.

A variety of compounds can be used to inhibit binding of leukotriene B4 to leukotriene B4 receptor by binding to leukotriene B4 receptor. Examples of such compounds include those which have the formula (“Formula I”):

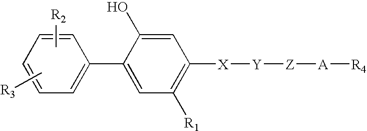

or a pharmaceutically acceptable salt or solvate thereof. R₁ is C₁-C₅ alkyl, C₂-C₅ alkenyl, C₂-C₅ alkynyl, C₁-C₄ alkoxy, (C₁-C₄ alkyl)thio, halo, or R₂-substitutedphenyl. Each of R₂ and R₃ is independently hydrogen, halo, hydroxy, C₁-C₄ alkyl, C₁-C₄ alkoxy, (C₁-C₄ alkyl)-S(O)_{q}—, trifluoromethyl, or di-(C₁-C₃ alkyl)amino. X is —O—, —S—, —C(═O)—, or —CH₂—; and Y is —O— or —CH₂—; or, when taken together, —X-Y— is —CH═CH— or —C≡C—. Z is a straight or branched chain C₁-C₁₀ alkylidenyl. A is a bond, —O—, —S—, —CH═CH—, or —CRₐR_{b}—, where each of Rₐ and R_{b} is independently hydrogen, C₃-C₅ alkyl, or R₇-substituted phenyl, or Rₐ and R_{b}, when taken together with the carbon atom to which they are attached, form a C₄-C₈ cycloalkyl ring. R₄ is a R₆ (as defined below), or R₄ is a moiety having one of the following formulae:

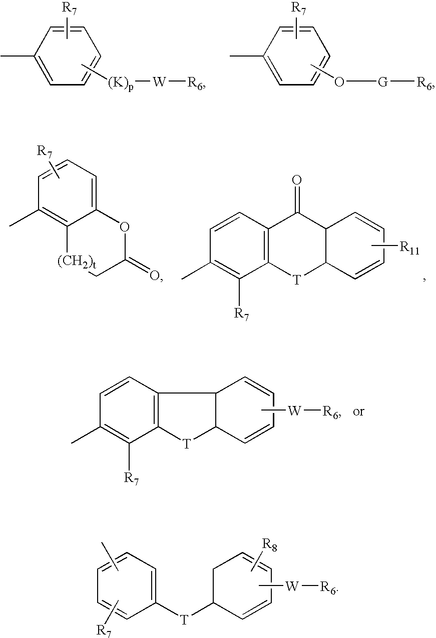

Each R₆ is independently —COOH, 5-tetrazolyl, —CON(R₉)₂, or —CONHSO₂R₁₀. Each R₇ is hydrogen, C₁-C₄ alkyl, C₂-C₅ alkenyl, C₂-C₅ alkynyl, benzyl, methoxy, —W—R₆, -T-G-R₆, (C₁-C₄ alkyl)-T-(C₁-C₄ alkylidenyl)-O—, or hydroxy. R₈ is hydrogen or halo. Each R₉ is independently hydrogen, phenyl, or C₁-C₄ alkyl; or R₉, when taken together with the nitrogen atom to which they are attached, form a morpholino, piperidino, piperazino, or pyrrolidino group. R₁₀ is C₁-C₄ alkyl or phenyl. R₁₁ is R₂, —W—R₆, or -T-G-R₆. Each W is a bond or straight or branched chain divalent hydrocarbyl radical of one to eight carbon atoms. Each G is a straight or branched chain divalent hydrocarbyl radical of one to eight carbon atoms. Each T is a bond, —CH₂—, —O—, —NH—, —NHCO—, —C(═O)—, or —S(O)_{q}— K is —C(═O)— or —CH(OH)—. Each q is independently 0, 1, or 2; p is 0 or 1; and t is 0 or 1. Preferably, when X is —O— or —S—, Y is not —O—; when A is —O— or —S—, R₄ is not R₆; when A is —O— or —S— and when Z is a bond, Y is not —O—; and, when p is 0, W is not a bond. The term “C₁-C₆ alkyl” refers to the straight and branched aliphatic radicals of 1 to 6 carbon atoms, such as methyl, ethyl, propyl, isopropyl, n-butyl, sec-butyl, tert-butyl, n-pentyl, 2,2-dimethylpropyl, hexyl, and the like. Included within this definition are the terms “C₁-C₃ alkyl”, “C₁-C₄ alkyl” and “C₁-C₅ alkyl”. The term “C₂-C₅ alkenyl”, refers to straight and branched aliphatic radicals of 2 to 5 carbon atoms containing one double bond, such as —CH═CH₂, —CH₂—CH═CH₂, —CH₂—CH₂—CH═CH₂, —CH—C(CH₃)═CH₂, —CH₂—CH═C(CH₃)₂, and the like. The term “C₂-C₅ alkynyl” refers to straight and branched aliphatic residues of 2 to 5 carbon atoms containing one triple bond, such as —C≡CH, —CH₂—C≡CH, —CH₂—CH₂—C≡CH, —CH₂—CH(CH₃) —C≡CH, —CH₂—C≡C—CH₃, and the like. The term “C₁-C₄ alkoxy” refers to, for example, methoxy, ethoxy, propoxy, isopropoxy, butoxy, sec-butoxy, and tert-butoxy. The term “halo” refers to, for example, fluoro, chloro, bromo, and iodo. The term or “C₁-C₁₀ alkylidenyl” refers to a divalent radical derived from a C₁-C₁₀ alkane such as —CH₂—, —CH(CH₃)—, —C(CH₃)₂—, —CH(C₂H₅)—, —CH₂—CH₂—, —CH₂—CH(CH₃)—, —CH(CH₃) —CH₂—, —CH(CH₃) —CH(CH₃)—, —CH₂—CH(C₂H₅)—, —CH₂—CH₂—CH₂—, —CH(CH₃) —CH₂—CH₂—, —CH₂—CH(CH₃) —CH₂—, —CH₂—CH(C₂H₅) —CH₂—, —CH₂—CH₂—CH(C₂H₅)—, —C(CH₃)₂—CH₂—CH₂—, —CH(CH₃) —CH₂—CH(CH₃)—, —CH₂—CH₂—CH₂—CH₂—, —CH₂—C(CH₃)₂—CH₂—CH₂—, —CH₂—C(CH₃)₂—CH₂—, —CH₂—CH₂—CH(C₂H₅) —CH₂—, —CH₂—CH₂—CH₂—CH₂—CH₂—, —CH(CH₃) —CH₂—CH₂—CH₂—CH₂—, —CH₂—CH₂—CH₂—CH₂—CH₂—CH₂—, —(CH₂)₁₀—, and the like. Included within this definition are the terms “C₁-C₄ alkylidene” and “C₂-C₄ alkylidene”. The term “C₄-C₈ cycloalkyl” refers to a cycloalkyl ring of four to eight carbon atoms, such as cyclobutyl, cyclopentyl, cyclohexyl, 4,4-dimethylcyclohexyl, cycloheptyl, cyclooctyl, and the like. The term “straight or branched chain divalent hydrocarbyl residue of one to eight carbon atoms” refers to a divalent radical derived from a straight or branched alkane, alkene, or alkyne of one to eight carbon atoms. Depending upon the branching and number of carbon atoms, as will be appreciated by organic chemists, such a moiety can contain one, two, or three double or triple bonds, or combinations of both. As such, this term can be considered an alkylidene group as defined above containing from 1 to 8 carbon atoms optionally containing one to three double or triple bonds, or combinations of the two, limited as noted in the preceding sentence.

As indicated above, pharmaceutically acceptable base addition salts of the compounds of Formula I can also be used. Such salts include those derived from inorganic bases, such as ammonium and alkali and alkaline earth metal hydroxides, carbonates, bicarbonates, and the like, as well as salts derived from basic organic amines, such as aliphatic and aromatic amines, aliphatic diamines, hydroxy alkylamines, and the like. Such bases useful in preparing the salts useful in the practice of the present invention thus include ammonium hydroxide, potassium carbonate, sodium bicarbonate, calcium hydroxide, methyl amine, diethyl amine, ethylene diamine, cyclohexylamine, ethanolamine, and the like. The potassium and sodium salt forms are particularly preferred.

This invention includes using both mono-salt forms, i.e., a 1:1 ratio of a compound of Formula I with a base as previously described, as well as di-salt forms in those instances where a compound of Formula I has two acidic groups. In addition, the method of the present invention can be practiced using solvate forms of the compounds of Formula I or salts thereof, such as ethanol solvates, hydrates, and the like.

It is recognized that in compounds having branched alkyl, alkylidenyl, or hydrocarbyl functionality, and in those compounds bearing double or triple bonds, various stereoisomeric products may exist. The method of the present invention is not limited to any particular stereoisomer but includes all possible individual isomers and mixtures thereof. The term “5-tetrazolyl” refers to both tautomers, i.e., (1H)-5-tetrazolyl and (2H)-5-tetrazolyl.

Illustrative compounds which can be used in the practice of the method of the present invention include 2-(2-propyl-3-(3-(2-ethyl-4-(4-fluorophenyl)-5-hydroxyphenoxy)propoxy)phenoxy)benzoic acid, 3-(2-(3-(2-ethyl-4-(4-fluorophenyl)-5-hydroxyphenoxy) propoxy)-6-(4-carboxy-phenoxy)phenyl)propionic acid, 1-(4-(carboxy-methoxy)phenyl)-1-(1H-tetrazol-5-yl)-6-(2-ethyl-4-(4-fluorophenyl)-5-hydroxyphenoxy)hexane, 3-(4-(7-carboxy-9-oxo-3-(3-(2-ethyl-4-(4-fluorophenyl)-5-hydroxyphenoxy)-propoxy)-9H-xanthene))propanoic acid, 5-(3-(2-(1-carboxy)-ethyl)-4-(3-(2-ethyl-4-(4-fluorophenyl)-5-hydroxyphenoxy)propoxy)phenyl)-4-pentynoic acid, pharmaceutically acceptable salts or solvates thereof, or combinations of these compounds, salts, and/or solvates. Preferably, the present invention is practiced with 2-(2-propyl-3-(3-(2-ethyl-4-(4-fluorophenyl)-5-hydroxyphenoxy)propoxy)phenoxy)benzoic acid or a pharmaceutically acceptable salt or solvate thereof.

Compounds of Formula I can be synthesized by established methods, such as those set forth in U.S. Pat. No. 5,462,954 to Baker et al. and in U.S. Pat. No. 5,910,505 to Fleisch et al., which are hereby incorporated by reference.

Other compounds that can be used to inhibit binding of leukotriene B4 to leukotriene B4 receptor by themselves binding to leukotriene B4 receptor include other leukotriene B4 receptor antagonists, such as those described in U.S. Pat. No. 5,998,454 to Fleisch et al.; U.S. Pat. No. 5,914,340 to Fleisch et al.; U.S. Pat. No. 5,817,864 to Fleisch et al.; U.S. Patent No. to U.S. Pat. No. 6,037,377 to Anderskewitz et al.; Perchonock et al., “Synthesis and Structure-Activity Relationship Studies of a Series of 5-Aryl-4,6-dithianonanedioic Acids and Related Compounds: A Novel Class of Leukotriene Antagonists,” J. Med. Chem., 29:1442-1452 (1986); Gleason et al., “Communications to the Editor,” J. Med. Chem., 30:959-961 (1987); Konno et al., “Synthesis and Structure-activity Relationships of a Series of Substituted-phenylpropionic acids as a Novel Class of Leukotriene B4 Antagonists,” Adv. Prostaglandin Thromboxane Leukot. Res., 21A:411-414 (1991); Daines et al., “(E)-3-[[[[6-(2-carboxyethenyl)-5-[[8-(4-methoxyphenyl)octyl]oxy]-2-pyridinyl]-methyl]thio]methyl]benzoic Acid: A Novel High-affinity Leukotriene B4 Receptor Antagonist,” J. Med. Chem., 36(18):2703-2705 (1993) (published erratum appears in J. Med. Chem., 36(25):4130 (1993); Showell et al., “The Preclinical Pharmacological Profile of the Potent and Selective Leukotriene B4 Antagonist CP-195543,” J. Pharmacol. Exp. Ther., 285(3):346-954 (1998); and Hullot et al., “Synthesis and Biochemical/Pharmacological Profile of the Novel Leukotriene B4 Receptor Antagonist Sodium (1S*,3S*)-1-hydroxy-3-[(3R*S*,E)-3-hydroxy-7-phenyl-1-hepten-1-yl]-1-cyclohexane acetate,” Arzneimittelforschung, 47(1):51-58 (1997), which are hereby incorporated by reference.

Yet other compounds which can be used to inhibit binding of leukotriene B4 to leukotriene B4 receptor by binding to leukotriene B4 receptor include antibodies (monoclonal or polyclonal) as well as peptide drugs, such as mimotopes which recognize and bind to the leukotriene B4 receptor. Methods for making and using such antibodies and mimotopes are well known to those skilled in the art and are described below in relation to making antibodies and mimotopes which recognize and bind to leukotriene A4 hydrolase.

Alternatively, binding of leukotriene B4 to leukotriene B4 receptor can be inhibited by using compounds which bind to leukotriene B4 to form, for example, a complex which is not capable of binding to leukotriene B4 receptor. Preferably, the binding of the compound to leukotriene B4 is specific; i.e., the compound does not bind substantially to other biological materials that are present in the sample. For example, it is preferred that the IC₅₀ of the compound to leukotriene B4 be greater than about 1.1 times, more preferably greater than about 1.5 times, still more preferably greater than about 2 times, still more preferably greater than about 5 times, still more preferably greater than about 10 times the IC₅₀ of the compound to all other biological materials in the sample. The degree of binding of the compound to leukotriene B4 is preferably greater (i.e., has a higher IC₅₀) than the binding of leukotriene B4 to leukotriene B4 receptor, although the desired effect can be achieved in some circumstances even when the degree of binding of the compound to leukotriene B4 is less (i.e., has a lower IC₅₀) than the binding of leukotriene B4 to leukotriene B4 receptor.

Still alternatively, binding of leukotriene B4 to leukotriene B4 receptor can be inhibited by inhibiting the production of leukotriene B4. Production of leukotriene B4 can be inhibited, for example, by inhibiting one or more of the steps in the leukotriene B4 synthetic pathway.

For example, the compound can be one which inhibits the conversion of leukotriene A4 to leukotriene B4.

Illustratively, the conversion of leukotriene A4 to leukotriene B4 by can be inhibited by contacting the sample with a compound that binds to leukotriene A4 to form, for example, a complex which is not susceptible or is less susceptible (than leukotriene A4) to hydrolysis by leukotriene A4 hydrolase.

Also illustratively, the conversion of leukotriene A4 to leukotriene B4 by can be inhibited by contacting the sample with a compound that inhibits the activity of leukotriene A4 hydrolase.

As one skilled in the art will appreciate, the activity of leukotriene A4 hydrolase can be inhibited by contacting the sample with a compound that binds leukotriene A4 hydrolase. Such leukotriene A4 hydrolase inhibitors can be traditional small molecules or pharmaceutically acceptable salts thereof. Examples of such leukotriene A4 hydrolase inhibitors include 3-oxiranyl benzoic acids and derivatives thereof; Rhone-Poulenc Rorer RP-64996; and 2-amino-3-phenylpropane derivatives, such as (2S)-2-amino-3-phenylpropyl mercaptan, 2-amino-3-(4′-benzyloxy)phenylpropyl mercaptan, and (2S)-3-(4-(2-naphthylmethyloxy)phenyl)-1,2-diamino-propane HCl, and its corresponding 4′-benzyloxy derivative. Other leukotriene A4 hydrolase inhibitors are described in, for example, U.S. Pat. No. 5,990,326 to Djuric et al.; U.S. Pat. No. 5,990,148 to Isakson et al.; U.S. Pat. No. 5,455,271 to Yuan et al.; International Patent Publictaion Nos. WO96/11192 and WO96/1099; Evans et al., Prostaglandins, Leukotrienes and Medicine, 23:167-171 (1986); Yuan et al, “Isolation and Identification of Metabolites of Leukotriene A4 Hydrolase Inhibitor SC-57461 in Rats,” Drug Metab. Dispos., 24(10):1124-1133 (1996); Tsuji et al., “Effects of SA6541, a Leukotriene A4 Hydrolase Inhibitor, and Indomethacin On Carrageenan-induced Murine Dermatitis,” Eur. J. Pharmacol., 346(1):81-85 (1998); and Penning et al., “Structure-activity Relationship Studies On 1-[2-(4-Phenylphenoxy)ethyl]pyrrolidine (SC-22716), a Potent Inhibitor of Leukotriene A(4) (LTA(4)) Hydrolase,” J. Med. Chem., 43(4):721-735 (2000), which are hereby incorporated by reference.

Leukotriene A4 hydrolase inhibitors can also be in the form of peptide drug products. See, generally, Bevan et al., Trends in Biotechnology, 13(3):115-121 (1995); Sepetov et al., Proc. Nat'l Acad. Sci. USA, 92:5426-5430 (1995); O'Connor et al., Cancer Chemother. Pharmacol., 34:225-229 (1994); and Webber et al., J. Med. Chem., 36:733-746 (1993), which are hereby incorporated by reference.

Drugs, such as peptide drugs, can be made using various methods known in the art. One such method utilizes the development of epitope libraries and biopanning of bacteriophage libraries. Briefly, attempts to define the binding sites for various monoclonal antibodies have led to the development of epitope libraries. Parmley et al., Gene, 73:305-318 (1988) (“Parmley”), which is hereby incorporated by reference, describe a bacteriophage expression vector that could display foreign epitopes on its surface. This vector could be used to construct large collections of bacteriophage which could include virtually all possible sequences of a short (e.g. six-amino-acid) peptide. Parmley, which is hereby incorporated by reference, also describes biopanning, which is a method for affinity-purifying phage displaying foreign epitopes using a specific antibody. See, also, e.g., Parmley; Cwirla et al., Proc. Nat'l Acad. Sci. USA, 87:6378-6382 (1990); Scott et al., Science, 249:386-390 (1990); Christian et al., J. Mol. Biol., 227:711-718 (1992); and Smith et al., Methods in Enzymology, 217:228-257 (1993) (“Smith”), which are hereby incorporated by reference.

After the development of epitope libraries, Smith, which is hereby incorporated by reference, then suggested that it should be possible to use the bacteriophage expression vector and biopanning technique of Parmley, which is hereby incorporated by reference, to identify epitopes from all possible sequences of a given length. This led to the idea of identifying peptide ligands for antibodies by biopanning epitope libraries, which could then be used in vaccine design, epitope mapping, the identification of genes, and many other applications (Parmley; and Scott, Trends in Biochem. Sci., 17:241-245 (1992), which are hereby incorporated by reference).

Using epitope libraries and biopanning, researchers searching for epitope sequences found instead peptide sequences which mimicked the epitope, i.e., sequences which did not identify a continuous linear native sequence or necessarily occur at all within a natural protein sequence. These mimicking peptides are called mimotopes. In this manner, mimotopes of various binding sites/proteins have been found. LaRocca et al., Hybridoma,11:191-201 (1992), which is hereby incorporated by reference, describes the expression of a mimotope of the human breast epithelial mucin tandem repeat in Escherichia coli. Balass et al., Proc. Nat'l Acad. Sci. USA, 90:10638-10642 (1993) (“Balass”), which is hereby incorporated by reference, identifies a hexapeptide that mimics a conformation-dependent binding site of the acetylcholine receptor. Hobart et al., Proc. R. Soc. London. B, 252:157-162 (1993), which is hereby incorporated by reference, discloses the isolation of a mimotope that mimics the C6 epitope (the epitope for the sixth component of complement).

The sequences of these and other mimotopes, by definition, do not identify a continuous linear native sequence or necessarily occur in any way in a naturally-occurring molecule, e.g., a naturally occurring protein. The sequences of the mimotopes merely form a peptide which functionally mimics a binding site on a naturally-occurring protein. For example, the mimotope described in Balass mimics the binding site of the acetylcholine receptor.

Many of these mimotopes are short peptides. The availability of short peptides which can be readily synthesized in large amounts and which can mimic naturally-occurring sequences (i.e. binding sites) can be exploited in the method of the present invention.

For example, using this technique, mimotopes to a monoclonal antibody that recognizes leukotriene A4 hydrolase can be identified. The sequences of these mimotopes represent short peptides which can then be used in various ways, for example as peptide drugs that bind to leukotriene A4 hydrolase and decrease the activity of leukotriene A4 hydrolase. Once the sequence of the mimotope is determined, the peptide drugs can be chemically synthesized.

Antibodies to leukotriene A4 hydrolase represent another class of compounds that are useful for inhibiting the conversion of leukotriene A4 to leukotriene B4 by inhibiting the activity of leukotriene A4 hydrolase. “Antibodies”, as used herein are meant to include antibody fragments, such as Fab, Fab2, and Fd fragments, as well as humanized forms. Humanized forms of the antibodies may be generated using one of the procedures known in the art, such as chimerization. The antibody binds to leukotriene A4 hydrolase, decreasing activity of the leukotriene A4 hydrolase. Suitable antibodies include polyclonal antibodies and monoclonal antibodies.

Monoclonal antibodies that bind to leukotriene A4 hydrolase can be produced by hybridomas. A hybridoma is an immortalized cell line which is capable of secreting a specific monoclonal antibody.

In general, techniques for preparing polyclonal and monoclonal antibodies as well as hybridomas capable of producing the desired antibodies are well known in the art. See, e.g., Campbell, Monoclonal Antibody Technology: Laboratory Techniques in Biochemistry and Molecular Biology, Amsterdam, The Netherlands: Elsevier Science Publishers (1984) (“Campbell”); and St. Groth et al., J. Immunol. Methods., 35:1-21 (1980), which are hereby incorporated by reference. Any animal (mouse, rabbit, etc.) which is known to produce antibodies can be immunized with the antigenic leukotriene A4 hydrolase (or an antigenic fragment thereof). Methods for immunization are well known in the art. Such methods include subcutaneous or intraperitoneal injection of the enzyme. One skilled in the art will recognize that the amount of the enzyme used for immunization will vary based on the animal which is immunized, the antigenicity of the enzyme, and the site of injection.

The enzyme which is used as an immunogen may be modified or administered in an adjuvant in order to increase the enzyme's antigenicity. Methods of increasing the antigenicity of an enzyme are well known in the art and include, but are not limited to, coupling the antigen with a heterologous protein (such as a globulin or beta-galactosidase) or including an adjuvant during immunization.

For monoclonal antibodies, spleen cells from the immunized animals are removed, fused with myeloma cells, such as SP2/O-Ag 15 myeloma cells, and allowed to become monoclonal antibody producing hybridoma cells.

Any one of a number of methods well known in the art can be used to identify the hybridoma cell which produces an antibody with the desired characteristics. These include screening the hybridomas using, for example, an ELISA assay, a western blot analysis, or a radioimmunoassay. See, e.g., Lutz et al., Exp. Cell Res., 175:109-124 (1988), which is hereby incorporated by reference.

Hybridomas secreting the desired antibodies are cloned, and the class and subclass are determined using procedures known in the art, such as those set described in Campbell, which is hereby incorporated by reference.

For polyclonal antibodies, antibody containing antisera is isolated from the immunized animal and is screened for the presence of antibodies with the desired specificity using one of the above-described procedures.

Also illustratively, the conversion of leukotriene A4 to leukotriene B4 by can be inhibited by contacting the sample with a nucleic acid compound that binds to a nucleic acid molecule encoding the leukotriene A4 hydrolase, thus decreasing expression of the leukotriene A4 hydrolase.

Suitable nucleic acid molecules include, for example, antisense nucleic acid molecules and ribozymes. Antisense nucleic acid molecules are complementary to at least a portion of the mRNA encoding leukotriene A4 hydrolase. Nucleic acid and amino acid sequences of leukotriene A4 hydrolases are known. See, for example, Medina et al., “Molecular Cloning and Expression of Mouse Leukotriene A-4 Hydrolase cDNA,” Biochem. Biophys. Res. Commun., 176:1516-1524 (1991); Minami, et al., “Molecular Cloning of a cDNA Coding for Human Leukotriene A-4 Hydrolase: Complete Primary Structure of an Enzyme Involved in Eicosanoid Synthesis,” J. Biol. Chem., 262:13873-13876 (1987); Funk et al., “Molecular Cloning and Amino Acid Sequence of Leukotriene Hydrolase,” Proc. Nat'l Acad. Sci. USA, 84:6677-6681 (1987); Mancini et al., “Cloning and Characterization of the Human Leukotriene A4 Hydrolase Gene,” Eur. J. Biochem., 231(1):65-71 (1995); Makita et al., “Molecular Cloning and Functional Expression of Rat Leukotriene A4 Hydrolase Using the Polymerase Chain Reaction,” FEBS Lett., 299(3):273-277 (1992); GenBank Accession No. M63848 (Mouse mRNA and amino acid sequences), GenBank Accession Nos. J03459, J02959, and U27293 (Human mRNA and amino acid sequences); and GenBank Accession No. S87522 (Rat mRNA and amino acid sequences), which are hereby incorporated by reference. Antisense nucleic acid molecules can be RNA or single-stranded DNA and can be complementary to the entire mRNA molecule encoding leukotriene A4 hydrolase (i.e., of the same nucleotide length as the entire molecule). It may be desirable, however, to work with a shorter molecule. In this instance, the antisense molecule can be complementary to a portion of the entire mRNA molecule encoding the leukotriene A4 hydrolase. These shorter antisense molecules are capable of hybridizing to the mRNA encoding the entire molecule and, preferably, consist of at least fifteen nucleotides and up to about 100 nucleotides. These antisense molecules can be used to reduce levels of leukotriene A4 hydrolase, by contacting the cells with an RNA or single-stranded DNA molecule that is complementary to at least a portion of the mRNA of the leukotriene A4 hydrolase (e.g., by introducing an antisense molecule into the sample). The antisense molecule can base-pair with the mRNA of the leukotriene A4 hydrolase, preventing translation of the mRNA into protein. Thus, an antisense molecule to the leukotriene A4 hydrolase can prevent translation of mRNA encoding the leukotriene A4 hydrolase into a functional leukotriene A4 hydrolase, thereby decreasing the activity of leukotriene A4 hydrolase in the cell.

Antisense molecules are contacted with the cells by any suitable means. In accordance with the methods of the subject invention, the cells are adenocarcinoma cancer cells, such as prostate cancer cells, lung cancer cells, stomach cancer cells, breast cancer cells, colon cancer cells, and pancreatic cancer cells. In one embodiment, the antisense RNA molecule is contacted with the cells by injecting the antisense RNA molecule directly into the cellular cytoplasm, where the RNA interferes with translation. A vector can also be used for introducing the antisense molecule into a cell. Such vectors include, for example, various plasmid and viral vectors. The antisense molecules could also be introduced into a cell using liposomes. For a general discussion of antisense molecules and their use, see Han et al., Proc. Nat'l Acad. Sci. USA, 88:4313-4317 (1991) (“Han”); and Rossi, British Medical Bulletin 51(1):217-225 (1995), which are hereby incorporated by reference.

A special category of antisense RNA molecules, known as ribozymes, having recognition sequences complementary to specific regions of the mRNA encoding the leukotriene A4 hydrolase, can also be used to inhibit the activity of leukotriene A4 hydrolase. Ribozymes not only complex with target sequences via complementary antisense sequences but also catalyze the hydrolysis, or cleavage, of the template mRNA molecule.

Expression of a ribozyme in a cell can inhibit gene expression (such as the expression of the leukotriene A4 hydrolase). More particularly, a ribozyme having a recognition sequence complementary to a region of a mRNA encoding the leukotriene A4 hydrolase can be used to decrease expression of leukotriene A4 hydrolase. A cell with a first level of expression of leukotriene A4 hydrolase is selected, and, then, the ribozyme is introduced into the cell. The ribozyme in the cell decreases expression of leukotriene A4 hydrolase in the cell, typically because mRNA encoding the leukotriene A4 hydrolase is cleaved and cannot be translated.

Ribozymes can be contacted with the cancer cells in accordance with the methods of the present invention by any suitable means. For example, the ribozyme can be injected directly into the cellular cytoplasm, where the ribozyme cleaves the mRNA and thereby interferes with translation. Alternatively, a vector can be used to introduce the ribozyme into a cell. Such vectors include various plasmid and viral vectors. In this regard, it should be noted that the DNA encoding the ribozyme does not need to be “incorporated” into the genome of the host cell; instead, for example, the ribozyme-encoding DNA molecule could be expressed in a host cell infected by a viral vector, with the vector expressing the ribozyme. The ribozyme molecules could also be introduced into a cell using liposomes. For a general discussion of ribozymes and their use, see, for example, Sarver et al., Science, 247:1222-122S (1990); Chrisey et al., Antisense Research and Development, 1(1):57-63 (1991); Rossi et al., AIDS Research and Human Retroviruses, 8(2):183-189 (1992); and Christoffersen et al., Journal of Medicinal Chemistry, 38(12):2023-2037 (1995), which are hereby incorporated by reference.

As indicated, some of these methods of transforming a cell are optimally carried out using an intermediate plasmid vector. U.S. Pat. No. 4,237,224 to Cohen et al., which is hereby incorporated by reference, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture. The DNA sequences are cloned into the plasmid vector using standard cloning procedures known in the art, as described by Sambrook et al., Molecular Cloning: A Laboratory Manual, 2nd Edition, Cold Spring Harbor, N.Y.: Cold Spring Harbor Laboratory Press,(1989), which is hereby incorporated by reference.

As indicated above, levels of leukotriene A4 hydrolase in an adenocarcinoma cancer cell, such as a prostate, lung, stomach, breast, colon, or pancreatic cancer cell, can be decreased by introducing an antisense or ribozyme construct into the cell. An antisense construct blocks translation of mRNA encoding leukotriene A4 hydrolase into the leukotriene A4 hydrolase. A ribozyme construct cleaves the mRNA encoding the leukotriene A4 hydrolase thus also prevents expression of functional leukotriene A4 hydrolase. For decreasing of expression of leukotriene A4 hydrolase in vivo, various gene therapy techniques can be utilized to introduce the antisense or ribozyme construct into the desired cell. The construct may need to be targeted to the desired cells (e.g., the prostate, lung, stomach, pancreatic, colon, or breast cancer cells) by known methods, since, in other cells of the subject, decreased expression of leukotriene A4 hydrolase may not be desired.

As indicated above, binding of leukotriene B4 to leukotriene B4 receptor can be inhibited by inhibiting the production of leukotriene B4. Also as indicated above, production of leukotriene B4 can be inhibited, for example, by inhibiting one or more of the steps in the leukotriene B4 synthesis pathway. Another method for inhibiting the leukotriene B4 synthesis pathway involves inhibiting the production of leukotriene A4, for example, by contacting the sample of adenocarcinoma cancer cells with a 5-lipoxygenase inhibitor. As used herein, 5-lipoxygenase inhibitor means any compound that directly or indirectly inhibits the activity of 5-lipoxygenase. Examples of such 5-lipoxygenase inhibitors include inhibitors of 5-lipoxygenase-activating protein (e.g., 3-(1-(4-chlorobenzyl)-3-t-butyl-thio-5-isopropyl-2-yl)-2,2-dimethylpropanoic acid and/or nordihydroguaiaretic acid) and nucleic acid molecules which decrease expression of 5-lipoxygenase. Further details with regard to these compounds are discussed in applicant's copending U.S. patent application Ser. No. 09/111,343, which is hereby incorporated by reference.

Alternatively, the method of the present invention can be practiced with compounds that inhibit the binding of leukotriene B4 to leukotriene B4 receptor but that do not inhibit the production of leukotriene A4. For example, the method of the present intention can be practiced with compounds that are not 5-lipoxygenase inhibitors, that are not inhibitors of 5-lipoxygenase-activating protein, that are not 3-(1-(4-chlorobenzyl)-3-t-butyl-thio-5-isopropyl-2-yl)-2,2-dimethylpropanoic acid, and/or that are not nordihydroguaiaretic acid.

As indicated above, the method of the present invention is carried out with a compound which inhibits binding of leukotriene B4 to leukotriene B4 receptor. This can be carried out, alternatively or additionally to the various methods set forth above, by decreasing expression of leukotriene B4 receptor. The methods discussed above with respect to decreasing expression of leukotriene A4 hydrolase are equally applicable here. More particularly, the compound can be a nucleic acid molecule which binds to a nucleic acid molecule encoding leukotriene B4 receptor, such as, for example, antisense nucleic acid molecules and ribozymes targeted to the nucleic acid molecule encoding leukotriene B4 receptor. The nucleic acid and amino acid sequences of leukotriene B4 receptor, which can be used is to design suitable antisense nucleic acid molecules and ribozymes are disclosed in, for example, Martin et al., “Leukotriene Binding, Signaling, and Analysis of HIV Coreceptor Function in Mouse and Human Leukotriene B4 Receptor-transfected Cells,” J. Biol. Chem., 274(13):8597-8603 (1999); Yokomizo et al., “A G-protein-coupled Receptor for Leukotriene B4 That Mediates Chemotaxis,” Nature, 387(6633):620-624 (1997); Toda et al., “Cloning and Characterization of Rat Leukotriene B(4) Receptor,” Biochem. Biophys. Res. Commun., 262(3):806-812 (1999); GenBank Accession No. AF077673 (mouse gene and amino acid sequences); GenBank Accession Nos. D89078 and D89079 (human gene and amino acid sequences); and GenBank Accession No. AB025230 (rat gene and amino acid sequences), which are hereby incorporated by reference.

Having now identified various compounds which can be used to inhibit binding of leukotriene B4 to leukotriene B4 receptor, the method of the subject invention is based on the finding that these compounds decrease proliferation of adenocarcinoma cancer cells, and/or induce apoptosis of adenocarcinoma cancer cells, and/or induce differentiation of adenocarcinoma cancer cells into non-cancerous cells. The meaning of the terms “proliferation”, “apoptosis”, and “differentiation” are readily understood in the art. Illustrative methods for assaying for proliferation, apoptosis, or differentiation are provided in the examples which follow and are also described in applicant's copending U.S. patent application Ser. No. 09/111,343, which is hereby incorporated by reference. These methods could be used to identify other compounds which inhibit binding of leukotriene B4 to leukotriene B4 receptor and which “decrease” proliferation, “induce” apoptosis, and/or “induce” differentiation.

The above discussion describes the concept of the subject invention in relation to the effect of compounds which inhibit binding of leukotriene B4 to leukotriene B4 receptor on adenocarcinoma cancer cells, especially cancerous epithelial cells, such as prostate cancer cells, lung cancer cells, stomach cancer cells, breast cancer cells, pancreatic cancer cells, and colon cancer cells. The methods of the present invention can be practiced in vitro or in vivo.

For example, the method of the present invention can be used in vitro to screen for compounds which are potentially useful in treating adenocarcinoma cancer (such as prostate, lung, stomach, breast, colon, and/or pancreatic cancer); to evaluate a compound's efficacy in treating adenocarcinoma cancer; or to investigate the mechanism by which a compound combats adenocarcinoma cancer (e.g., whether it does so by inducing apoptosis, by inducing differentiation, by decreasing proliferation, etc). For example, once a compound has been identified as a compound that inhibits binding of leukotriene B4 to leukotriene B4 receptor, one skilled in the art can apply the method of the present invention in vitro to evaluate the degree to which the compound induces apoptosis, induces differentiation, and/or decreases proliferation of cancer cells; or one skilled in the art can apply the method of the present invention to determine whether the compound operates by inducing apoptosis, by inducing differentiation, by decreasing proliferation, or by a combination of these methods.

Alternatively, the method of the present invention can be used in vivo to treat adenocarcinoma cancers, such as prostate cancer, lung cancer, stomach cancer, pancreatic cancer, breast cancer, and colon cancer. In the case where the method of the present invention is carried out in vivo, for example, where the adenocarcinoma cancer cells are present in a human subject, contacting can be carried out by administering a therapeutically effective amount of the compound to the human subject, for example, by directly injecting the compound into a tumor. Details with regard to administering compounds in accordance with the method of the present invention are described below.

The present invention, in another aspect thereof, relates to a method of treating adenocarcinomas, such as prostate cancer, lung cancer, stomach cancer, breast cancer, pancreatic cancer, colon cancer, or other cancers involving epithelial cells, in a subject. The method includes administering to the subject an amount of a compound effective to inhibit binding of leukotriene B4 to leukotriene B4 receptor.

Suitable subjects include, for example mammals, such as rats, mice, cats, dogs, monkeys, and humans. Suitable human subjects include, for example, those which have previously been determined to be at risk of having prostate cancer, lung cancer, stomach cancer, pancreatic cancer, colon cancer, and/or breast cancer and those who have been diagnosed as having prostate cancer, lung cancer, stomach cancer, pancreatic cancer, colon cancer, and/or breast cancer. Preferably, the subject does not suffer from oral squamous cell carcinoma or would not otherwise be indicated as a candidate for treatment of oral squamous cell carcinoma. In subjects who are determined to be at risk of having adenocarcinoma (and, preferably, who do not suffer from oral squamous cell carcinoma), the compounds which inhibit binding of leukotriene B4 to leukotriene B4 receptor are administered to the subject preferably under conditions effective to decrease proliferation and/or induce apoptosis and/or induce differentiation of the adenocarcinoma cancer cells in the event that they develop. Such preventive (which is not used in the absolute 100% sense) therapy can be useful in high risk individuals as long as the adverse side effects of the administration of compounds which inhibit binding of leukotriene B4 to leukotriene B4 receptor are outweighed by the potential benefit of prevention.

Any of the compounds described above can be used in the treatment method of the present invention. For example, compounds which inhibit binding of leukotriene B4 to leukotriene B4 receptor, such as traditional chemicals and peptide drugs disclosed herein that bind directly to leukotriene B4 receptor, may be administered alone or in combination with compatible carriers as a composition. Compatible carriers include suitable pharmaceutical carriers or diluents. The diluent or carrier ingredients should be selected so that they do not diminish the therapeutic effects of the compounds used in the present invention.

The compositions herein may be made up in any suitable form appropriate for the desired use. Examples of suitable dosage forms include oral, parenteral, or topical dosage forms.

Suitable dosage forms for oral use include tablets, dispersible powders, granules, capsules, suspensions, syrups, and elixirs. Inert diluents and carriers for tablets include, for example, calcium carbonate, sodium carbonate, lactose, and talc. Tablets may also contain granulating and disintegrating agents, such as starch and alginic acid; binding agents, such as starch, gelatin, and acacia; and lubricating agents, such as magnesium stearate, stearic acid, and talc. Tablets may be uncoated or may be coated by known techniques to delay disintegration and absorption. Inert diluents and carriers which may be used in capsules include, for example, calcium carbonate, calcium phosphate, and kaolin. Suspensions, syrups, and elixirs may contain conventional excipients, for example, methyl cellulose, tragacanth, sodium alginate; wetting agents, such as lecithin and polyoxyethylene stearate; and preservatives, such as ethyl-p-hydroxybenzoate.

Dosage forms suitable for parenteral administration include solutions, suspensions, dispersions, emulsions, and the like. They may also be manufactured in the form of sterile solid compositions which can be dissolved or suspended in sterile injectable medium immediately before use. They may contain suspending or dispersing agents known in the art. Examples of parenteral administration are intraventricular, intracerebral, intramuscular, intravenous, intraperitoneal, rectal, and subcutaneous administration.

In addition to the above, generally non-active components of the above-described formulations, these formulations can include other active materials, particularly, actives which have been identified as useful in the treatment of prostate, lung, stomach, breast, colon, pancreatic cancers and/or other adenocarcinomas. These actives can be broad-based anti-cancer agents, such that they also are useful in treating other types of cancers (i.e., in addition to adenocarcinomas) or they may be more specific, for example, in the case where the other active is useful for treating adenocarcinomas but not useful for treating oral squamous cell carcinoma. The other actives can also have non-anti-cancer pharmacological properties in addition to their anti-andenocarcinoma properties. For example, the other actives can have anti-inflammatory properties, or, alternatively, they can have no such anti-inflammatory properties.

It will be appreciated that the actual preferred amount of compound to be administered according to the present invention will vary according to the particular compound, the particular composition formulated, and the mode of administration. Many factors that may modify the action of the compound (e.g., body weight, sex, diet, time of administration, route of administration, rate of excretion, condition of the subject, drug combinations, and reaction sensitivities and severities) can be taken into account by those skilled in the art. Administration can be carried out continuously or periodically within the maximum tolerated dose. Optimal administration rates for a given set of conditions can be ascertained by those skilled in the art using conventional dosage administration tests.

The present invention is further illustrated with the following examples.

### EXAMPLES

### Example 1

### Materials and Methods

The following materials and methods were used in the examples which follow.

Materials. DMEM, MEM, and McCoy's 5A media, penicillin-streptomycin solution, trypsin-EDTA solution, proteinase K, propidium iodide, RNAse A, and phenol/chloroform were purchased from Sigma Chemicals (St. Louis, Mo.). Fetal bovine serum (“FBS”) was obtained from Atlanta Biologicals (Norcross, Ga.). APO-BRDU kits for TUNEL assay were from purchased from Pharmingen (San Diego, Calif.). Leukotriene B4 (“LTB4”) was obtained from Cayman Chemicals (Ann Arbor, Mich.). The LTB4 antagonist, 2-(2-propyl-3-(3-(2-ethyl-4-(4-fluorophenyl)-5-hydroxyphenoxy)propoxy)-phenoxy)benzoic acid (hereinafter “LY293111”), was provided by Eli Lilly and Company (Indianapolis, Ind.). The LTD4 antagonist, 1-(2-hydroxy-3-propyl-4-(4-(1H-tetrazol-5-yl)butoxy)phenyl)ethanone, (hereinafter, “LY171883”), was purchased from Biomol Research Laboratories (Plymouth Meeting, Pa.).

Cell culture. The following pancreatic cancer cell lines were used: MiaPaCa-2 and PANC-1 (poorly-differentiated); Capan-1 and Capan-2 (well-differentiated); and HPAF and AsPC-1 (pleomorphic). These were purchased from American Type Culture Collection (Rockville, Md.). PANC-1 and MiaPaCa-2 were grown in DMEM media; HPAF and AsPC-1 were grown in MEM media; and Capan-1 and Capan-2 were grown in-McCoy's media. Media were supplemented with 10% FBS, and the cells were grown as monolayers in a humidified atmosphere of 95% O₂ and 5% CO₂ at 37° C. The cells were regularly seeded into 75 cm² flask with media changed every other day. For experiments, cells were grown to 80% confluence, digested with trypsin-EDTA, and plated either in 12 or 24-well plates at a concentration of 50,000/ml, as appropriate.

DNA synthesis by [³H]-Thymidine incorporation. Cells were plated in 12 or 24-well plates. After reaching 70% confluence, they were incubated in serum-free media for 24 hours, which was then replaced with fresh serum-free media with or without the appropriate concentration of leukotriene B4 receptor antagonist LY293111 (62.5-1000 nM), LTB4 (100-400 nM), or both. After the appropriate period of culture, cellular DNA synthesis was assayed by adding 0.5 μCi/well of [³H]-methyl-thymidine and incubating for another 2 hours. Then, the cells were washed twice with PBS, fixed with 10% trichloroacetic acid (“TCA”) for 30 minutes, and solubilized by adding 0.4 M NaOH to each well. Radioactivity, indicating incorporation of ³H-thymidine into DNA, was measured by adding a scintillation cocktail and counting on a scintillation counter (LKB RackBeta, Wallac, Turku, Finland)

Cell proliferation assay. Pancreatic cancer cells were seeded into 24-well microplates and incubated at 37° C. After 24 hours, cells were cultured in serum-free media with or without 250 nM LY293111 for 24, 48, and 72 hours. At the end of each time period, the cells were trypsinized to produce a single cell suspension, and the cell number in each well was determined using a Zi-Coulter Counter (Luton, Bedfordsire, United Kingdom).

Morphological changes using light microscope. Pancreatic cancer cells grown in 75 cm² flasks were treated with different concentrations of LY293111 for different periods of time. They were then viewed by phase-contrast microscopy, and photographs were taken.

DNA fragmentation assay. For analysis of DNA fragmentation by agarose gel electrophoresis, treated pancreatic cancer cells were detached from 75 cm² flask with trypsin-EDTA, washed twice with PBS, resuspended in 1 ml lysis buffer (25 mM EDTA, 10 mM Tris, 0.5% Triton X-100, and 1% SDS), and freeze-thawed at −80° C. overnight. Then, an equal volume of PEG/NaCl stock solution was added to the lysates to give a final concentration of 0.25% PEG and 1 M NaCl, and the tubes were centrifuged at 13,200 g at 4° C. for 30 minutes. The supernatants containing fragmented small molecular weight DNA were then incubated with RNAse A (0.3 mg/ml) at 37° C. for one hour and then with 10 ml proteinase K (10 mg/ml) for three hours. Samples were then extracted with phenol-chloroform and precipitated with 70% ethanol overnight. The pellets containing DNA were then resuspended in 50 ml of TE buffer, subjected to electrophoresis on a 1.8% agarose gel containing ethidium bromide (1 mg/ml), visualized by UV illumination, and analyzed using a Molecular Analyst Gel Documentation system (Bio-rad Laboratories, Hercules, Calif.).

TUNEL assay. Apoptosis was measured by flow cytometry using a terminal deoxynucleotidyl transferase-mediated deoxyuridine triphosphate nick-end labeling (“TUNEL”) assay. Cells were treated with LY293111 for the appropriate period of time, then digested with trypsin-EDTA, washed with ice-cold PBS twice, and fixed in 10% paraformaldehyde on ice for 20 minutes. Cells were then washed with PBS, permeabilized with 70% ethanol for at least 4 hours, washed again with PBS and incubated with 2.5 units of terminal deoxynucleotidyl transferase (TdT enzyme) and 100 pmol Br-dUTP in DNA labeling solution for one hour at 37° C. Cells were then rinsed twice, resuspended in 0.1 ml fluorescein labeled anti-BrdU antibody solution in the dark for 30 minutes. Then 0.5 ml propidium iodide/RNase A solution was added, and the cells analyzed by flow cytometry at 488 nm excitation.

Statistical analysis. Data was analyzed by analysis of variance (“ANOVA”) with Dunnett's of Bonferroni's corrections for multiple comparisons, as appropriate. This analysis was performed with the Prism software package (GraphPad, San Diego, Calif.). Data are expressed as mean±SEM.

### Example 2

### Effect of LY293111 on Pancreatic Cancer Cells

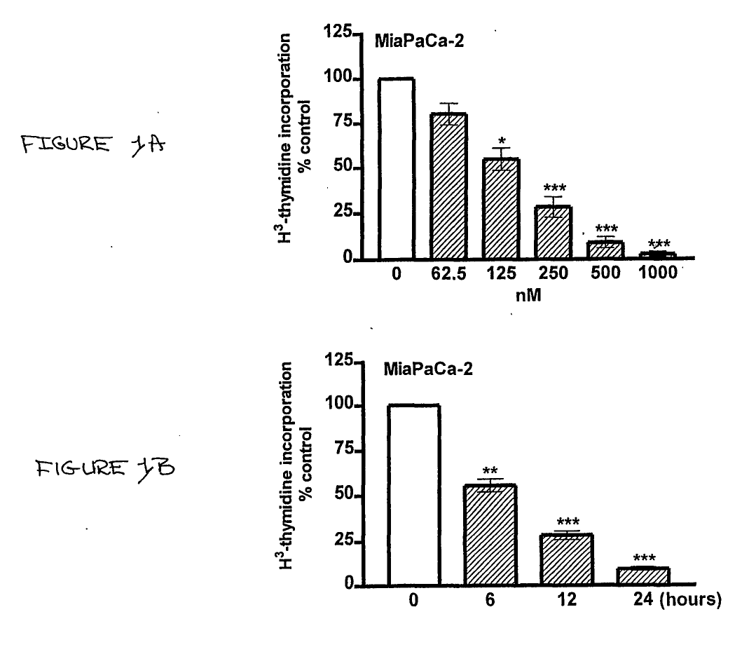

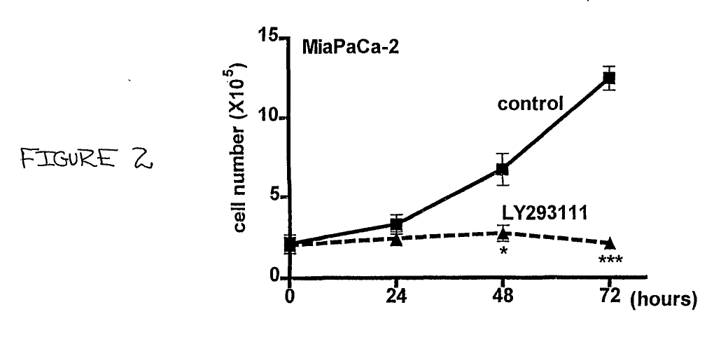

The leukotriene B4 receptor antagonist, LY293111 caused concentration-dependent (FIG. 1A) [F(5,30)=77.30, P<0.0001] and time-dependent (FIG. 1B) [F(3,20)=331.1, P<0.0001] inhibition of thymidine incorporation and cell number (FIG. 2) in MiaPaCa-2 pancreatic cancer cell line. Experiments carried out with 5 other pancreatic cancer cell lines (HPAF, Capan-1, Capan-2, AsPC-1, and PANC-1) also showed that LY293111 inhibited proliferation, in a concentration-dependent fashion, after 24 hours. The results of these experiments are presented in Table I, where results are expressed as percent of control (mean±SEM) and where *=P<0.05, **=P<0.01, and ***=P<0.001.

TABLE I
LY293111 (nM), 24 hours, % control

| concentration | 62.5 nM | 125 nM | 250 nM | 500 nM | 1000 nM |
|---|---|---|---|---|---|
| HPAF | 103.6 ± 21.8 | 97.6 ± 24.5 | 49.3 ± 9.0** | 10.3 ± 3.0*** | 2.3 ± 1.4*** |
| Capan-1 | 99.1 ± 4.8 | 56.9 ± 4.9** | 16.4 ± 2.0*** | 15.7 ± 3.4*** | 0.2 ± 0.3*** |
| Capan-2 | 95.4 ± 8.7 | 67.3 ± 6.8** | 51.2 ± 5.8** | 15.8 ± 9.6*** | 8.9 ± 2.1*** |
| AsPC-1 | 90.6 ± 4.6 | 72.8 ± 5.4* | 44.5 ± 7.7** | 18.4 ± 6.9*** | 7.3 ± 1.2*** |
| PANC-1 | 89.4 ± 4.4 | 65.2 ± 3.8** | 43.5 ± 5 7** | 12.4 ± 3.1*** | 5.6 ± 1.0*** |

In addition, the temporal effects of LY293111 on cell proliferation were investigated with these 5 other pancreatic cancer cell lines (HPAF, Capan-1, Capan-2, ASPC-1, and PANC-1). The results, presented in Table II, show that 500 nM LY293111 time-dependently inhibited pancreatic cancer cell proliferation of each of these cell lines. In Table II, the results are expressed as percent of control (mean±SEM), and *=P<0.05, **=P<0.01, and ***=P<0.001.

TABLE II
LY293111 (500 nM), % control

| time | 6 h | 12 h | 24 h |
|---|---|---|---|
| HPAF | 67.5 ± 11.6** | 35.7 ± 8.9*** | 12.4 ± 3.3*** |
| Capan-1 | 70.5 ± 8.7** | 44.5 ± 10.2** | 15.7 ± 3.4*** |
| Capan-2 | 57.8 ± 4.3** | 32.1 ± 5.6*** | 10.9 ± 6.0*** |
| AsPC-1 | 65.8 ± 7.7** | 44.7 ± 4.1** | 16.7 ± 2.1*** |
| PANC-1 | 60.2 ± 6.5** | 48.9 ± 5.3** | 13.6 ± 1.0*** |

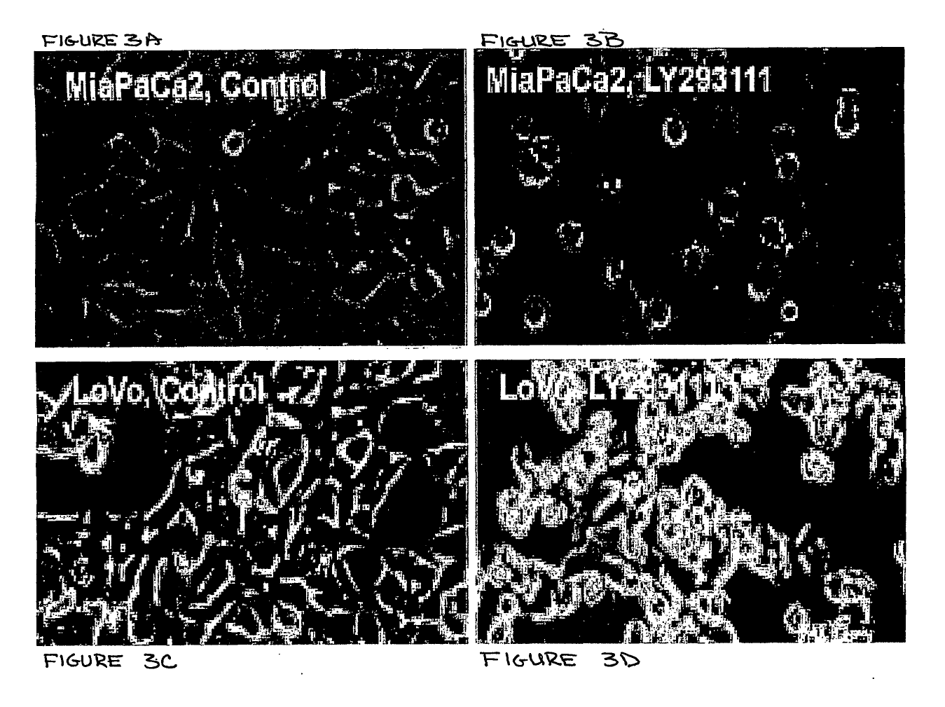

LY293111 inhibited the proliferation by at least 50% at a concentration of 250 nM and by more than 95% at 1000 nM. Phase-contrast microscopy revealed marked morphological changes in treated MiaPaCa-2 pancreatic cancer cells (FIG. 3B) compared with non-treated controls (FIG. 3A), as early as 4 hours after treatment with 250 nM LY293111. Over time, the treated cells became rounded and exhibited membrane blebbing, chromatin condensation, nuclear fragmentation and finally detached from the microplate. These morphological changes have been previously interpreted as reflecting apoptosis. Similar results were seen for colonic cell line LoVo, as shown in FIG. 3C (treated with LY293111) and FIG. 3D (control).

### Example 3

### Effect of LY171883 on Pancreatic Cancer Cells

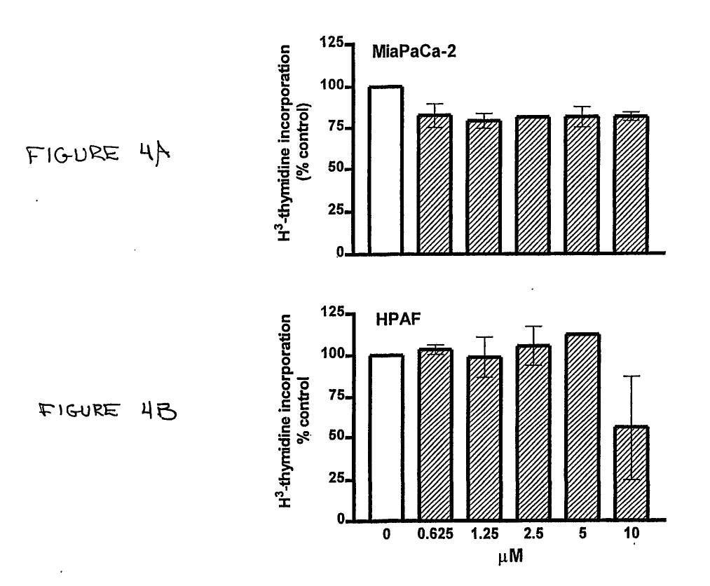

In contrast to the effects of the LTB4 antagonist, LY293111, a specific LTD4 receptor antagonist, LY171883, only affected proliferation at the highest concentration (10 mM) used in MiaPaCa-2 [F(5,6)=1.875, P=0.2316] and HPAF [F(5,6)=1.940, P=0.2217] pancreatic cancer cells. However, this is likely to reflect a toxic effect. The results for MiaPaCa-2 and HPAF pancreatic cancer cells are presented in FIGS. 4A and 4B, respectively.

### Example 4

### Effects of Leukotriene B4 on Pancreatic Cancer Cells Proliferation

Leukotriene B4 stimulated the proliferation of MiaPaCa-2 [F(3,8)=64.02 and F(3,8)=50.31, P<0.0001] and HPAF [F(3,8)=30.06,P<0.0001] pancreatic cancer cell lines in a both concentration and time-dependent manner. FIG. 5A shows the effects of different concentrations of LTB4 on thymidine incorporation in MiaPaCa-2 cells after 24 hours. FIGS. 5B and 5C show the effects of 100 nM of LTB4 on thymidine incorporation in MiaPaCa-2 cells (FIG. 5B) and HPAF cells (FIG. 5C) for 24, 48, and 72 hours. At a concentration of 100 nM for 72 hours, LTB4 more than doubled thymidine incorporation compared with control.

### Example 5

### Effect of LY293111 on the Stimulatory Effects of LTB4 on Pancreatic Cancer Cells Proliferation

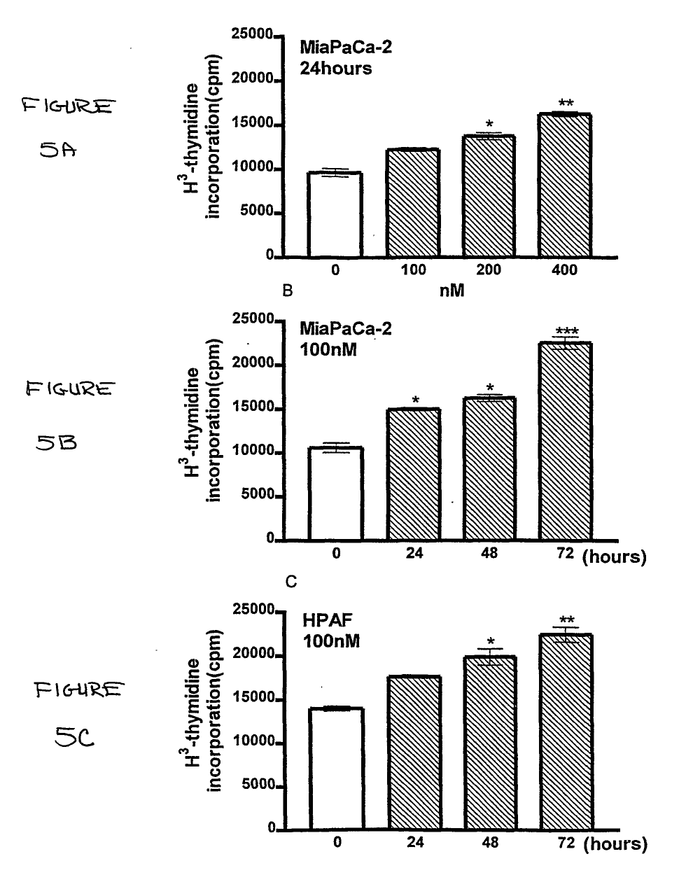

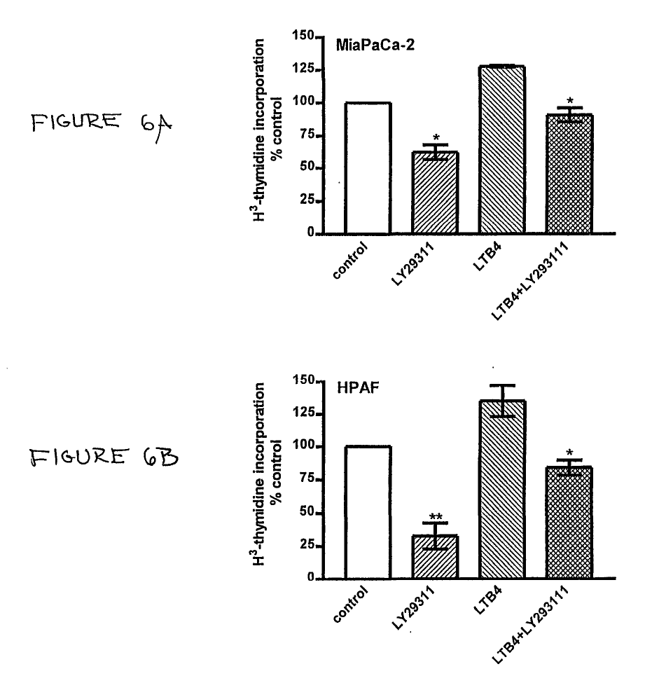

LTB4 abolished the inhibitory effect of the leukotriene B4 receptor antagonist, LY293111, on the proliferation of MiaPaCa-2 (FIG. 6A) and HPAF (FIG. 6B) pancreatic cancer cell lines. After 24 hours treatment with 100 nM LTB4, there was a 25% increase of proliferation compared with control, and LY293111 at 250 nM caused 50% decrease of proliferation. When treated with the combination of LTB4 and its antagonist, LY293111, proliferation was restored to almost the control level.

### Example 6

### DNA Fragmentation Assay

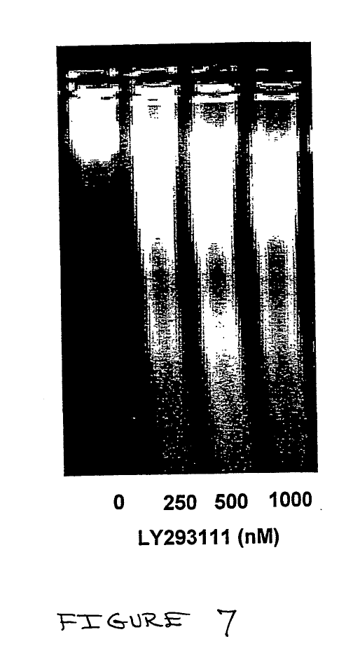

A DNA fragmentation assay was used to study apoptosis of pancreatic cancer cells treated with LY293111. After treatment of MiaPaCa-2 cells with 250, 500 and 1000 nM LY293111 for 48 hours, a distinctive DNA ladder was seen after running cellular DNA on 1.8% agarose gel, while no such ladder was seen in control cells (FIG. 7). Similar results were obtained in the other pancreatic cancer cell lines tested.

### Example 7

### TUNEL Assay

LY293111-induced apoptosis of pancreatic cancer cell lines was also evaluated using the TUNEL assay. Treatment of MiaPaCa-2 cells with 250 nM and 500 nM LY293111 for 24 hours greatly increased apoptosis to 12.6% and 18.6%, respectively. In contrast, the degree of apoptosis in the control cell population was very low (1.5%). The results are presented in FIGS. 8A-8C, where an increase of fluorescence events in the upper right quadrant is due to UTP labeling of fragmented DNA. Similar apoptosis was induced in other pancreatic, colonic, and breast cancer cell lines investigated.

### Example 8

### Effect of LY293111 on the Proliferation of Pancreatic and Colonic Cancer Cells

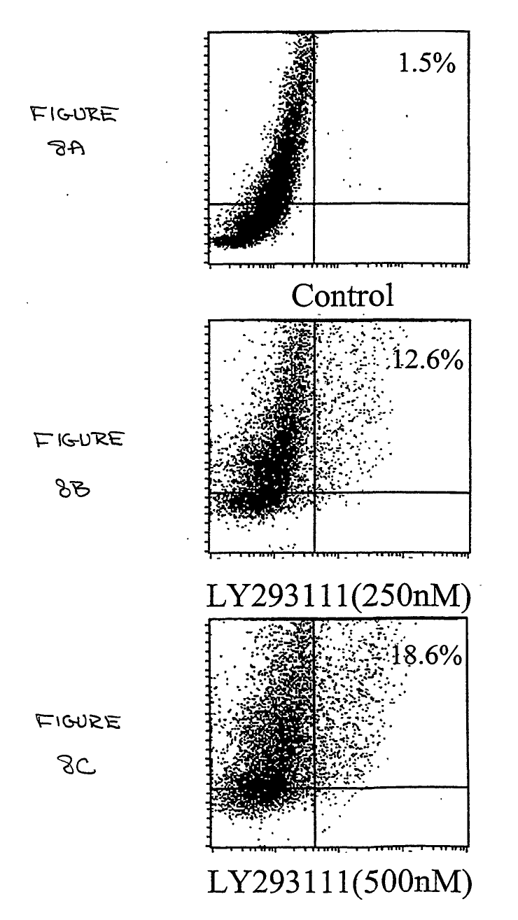

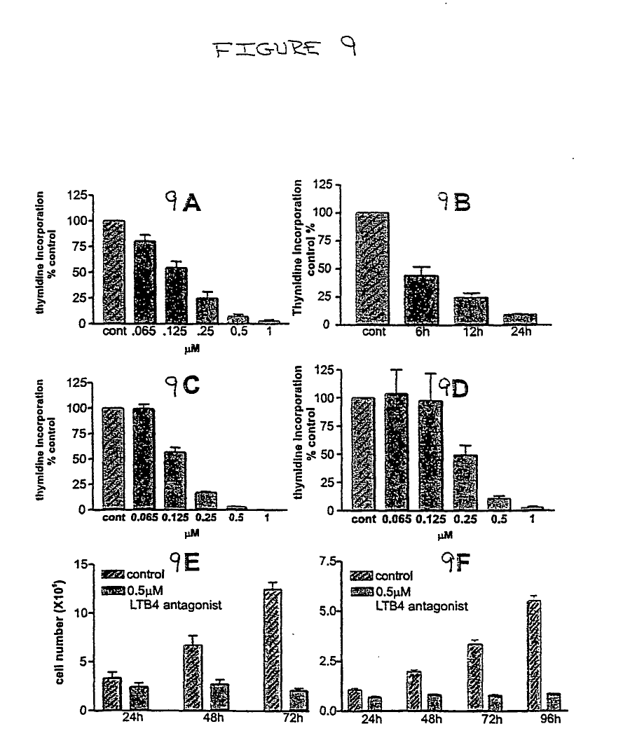

The effect of LY293111 on cancer cell proliferation, was investigated by measuring thymidine incorporation and cell number. LY293111 caused a profound concentration-dependent and time-dependent inhibition of proliferation of all pancreatic, colonic, and breast cancer cell lines studied. The results are presented in FIGS. 9A-9F. FIGS. 9A and 9B pertain to pancreatic cancer cell line MiaPaCa2; FIG. 9C pertains to pancreatic cancer cell line HPAF; and FIG. 9D pertains to pancreatic cancer cell line Capan2. Each of FIGS. 9A, 9C, and 9D show the dosage effects of LY293111 on these pancreatic cancer cell lines. In addition, FIG. 9B shows the time dependent effects of LY293111 on the pancreatic cancer cell line MiaPaCa2. FIGS. 9E and 9F shows the effect of 0.5 μM LY293111 on cell number vs control as a function of time for pancreatic cancer cell line MiaPaCa2 (FIG. 9E) and for colonic cancer cell line LoVo (FIG. 9F).

### Example 9

### Effect of LTB4 and LY293111 on Bcl-2

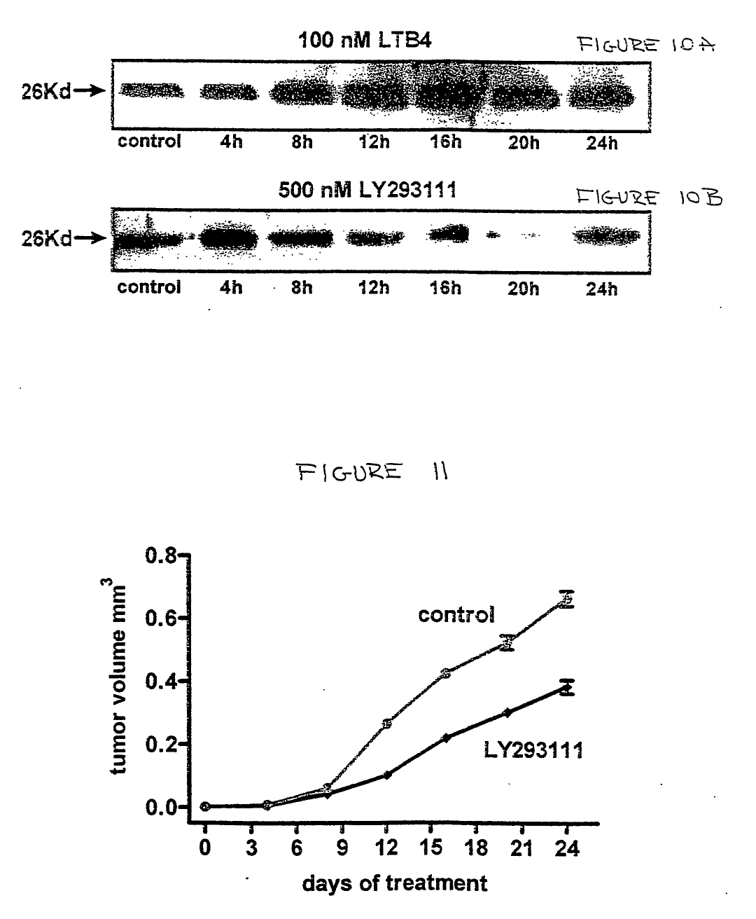

As shown in FIG. 10A, LTB4 (100 nM) was found to increase expression of the pro-apoptotic protein, Bcl-2, in MCF-7 breast cancer cells, as revealed by western blotting using Bcl-2 specific antisera. In contrast, as shown in FIG. 10B, LY293111 (500 nM) inhibited expression of the pro-apoptotic protein, Bcl-2, in MCF-7 breast cancer cells.

### Example 10

### Effect of LY293111 on Growth of AsPc-1 Human Pancreatic Tumor Cell Line Xenografted Into Athymic Mice

Athymic mice received a subcutaneous injection of 3 million cancer cells in 50 μl of serum-free media. The highly malignant and aggressive pancreatic tumor cell line, AsPc-1, was used in this experiment. The tumors were allowed to establish and grow for four days prior to treatment with 0.01% LY293111 in the drinking water. FIG. 11 shows the size of the tumor (in mm³) as a function of treatment time. The results show that mice receiving LY293111 in their drinking water had smaller tumors than those in the control group.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed is:

1. A method of decreasing proliferation of adenocarcinoma cancer cells, or of inducing apoptosis of adenocarcinoma cancer cells, or of inducing differentiation of adenocarcinoma cancer cells into non-cancerous cells, said method comprising:
contacting a sample comprising adenocarcinoma cancer cells with a compound under conditions effective to inhibit binding of leukotriene B4 to leukotriene B4 receptor.

2. A method according to claim 1, wherein the sample does not comprise oral squamous cell carcinoma cells.

3. A method according to claim 1, wherein the sample comprises prostate cancer cells, lung cancer cells, stomach cancer cells breast cancer cells, pancreatic cancer cells, colon cancer cells, or combinations thereof.

4. A method according to claim 1, wherein the compound inhibits binding of leukotriene B4 to leukotriene B4 receptor by binding to leukotriene B4 receptor.

5. A method according to claim 4, wherein the compound has the formula: 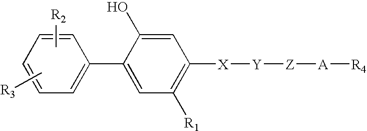
or a pharmaceutically acceptable salt or solvate thereof, wherein:
R₁ is C₁-C₅ alkyl, C₂-C₅ alkenyl, C₂-C₅ alkynyl, C—C₄ alkoxy, (C₁-C₄ alkyl)thio, halo, or R₂-substitutedphenyl;
each of R₂ and R₃ is independently hydrogen, halo, hydroxy, C₁-C₄ alkyl, C₁-C₄ alkoxy, (C₁-C₄ alkyl)-S(O)_{q}—, trifluoromethyl, or di-(C₁-C₃ alkyl)amino;
X is —O—, —S—, —C(═O), or —CH₂—, and Y is —O— or —CH₂—; or, when taken together, —X—Y— is —CH═CH— or —C≡C—;
Z is a straight or branched chain C₁-C₁₀ alkylidenyl;
A is a bond, —O—, —S—, —CH═CH—, or —CRₐR_{b}—, where each of Rₐ and R_{b} is independently hydrogen, C₁-C₁₀ alkyl, or R₇-substituted phenyl, or Rₐ and R_{b}, when taken together with the carbon atom to which they are attached, form a C₄-C₈ cycloalkyl ring;
R₄ is a R₆ or R₄ is a moiety having one of the following formulae: 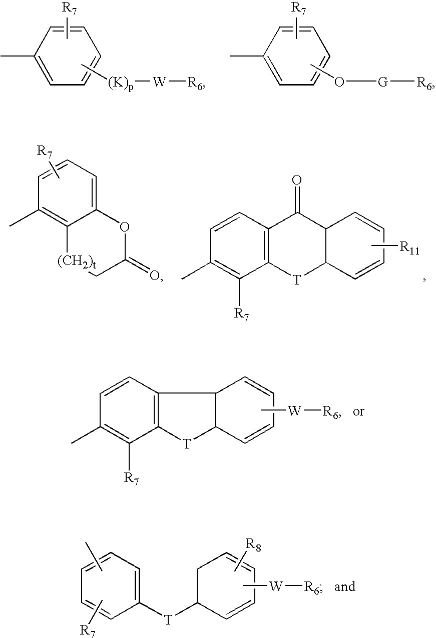
wherein:
each R₆ is independently —COOH, 5-tetrazolyl, —CON(R₉)₂, or —CONHSO₂R₁₀;
each R₇ is hydrogen, C₁-C₄ alkyl, C₂-C₅ alkenyl, C₂-C₅ alkynyl, benzyl, methoxy, —W—R₆, -T-G-R₆, (C₁-C₄ alkyl)-T-(C₂-C₄ alkylidenyl)-O—, or hydroxy;
R₈ is hydrogen or halo;
each R₉ is independently hydrogen, phenyl, or C₁-C₄ alkyl, or R₉, when taken together with the nitrogen atom to which they are attached, form a morpholino, piperidino, piperazino, or pyrrolidino group;
R₁₀ is C₁-C₄ alkyl or phenyl;
R₁₁ is R₂, —W—R₆, or -T-G-R₆;
each W is a bond or straight or branched chain divalent hydrocarbyl radical of one to eight carbon atoms;
each G is a straight or branched chain divalent hydrocarbyl radical of one to eight carbon atoms;
each T is a bond, —CH₂—, —O—, —NH—, —NHCO—, —C(═O)—, or —S(O)_{q}—;
K is —C(═O)— or —CH(OH)—;
each q is independently 0, 1, or 2;
p is 0 or 1; and
t is 0 or 1;
provided that, when X is —O— or —S—, Y is not —O—;
further provided that, when A is —O— or —S—, R₄ is not R₆;
further provided that, when A is —O— or —S— and Z is a bond, Y is not —O—; and
further provided that W is not a bond when p is 0.

6. A method according to claim 4, wherein the compound is selected from the group consisting of 2-(2-propyl-3-(3-(2-ethyl-4-(4-fluorophenyl)-5-hydroxyphenoxy)propoxy)phenoxy)benzoic acid, 3-(2-(3-(2-ethyl-4-(4-fluorophenyl)-5-hydroxyphenoxy)propoxy)-6-(4-carboxy-phenoxy)phenyl)propionic acid, 1-(4-(carboxy-methoxy)phenyl)-1-(1H-tetrazol-5-yl)-6-(2-ethyl-4-(4-fluorophenyl)-5-hydroxyphenoxy)hexane, 3-(4-(7-carboxy-9-oxo-3-(3-(2-ethyl-4-(4-fluorophenyl)-5-hydroxyphenoxy)-propoxy)-9H-xanthene))propanoic acid, 5-(3-(2-(1-carboxy)-ethyl)-4-(3-(2-ethyl-4-(4-fluorophenyl)-5-hydroxyphenoxy)-propoxy)phenyl)-4-pentynoic acid, a pharmaceutically acceptable salt or solvate thereof, and combinations thereof.

7. A method according to claim 4, wherein the compound is 2-(2-propyl-3-(3-(2-ethyl-4-(4-fluorophenyl)-5-hydroxyphenoxy)propoxy)phenoxy)benzoic acid or a pharmaceutically acceptable salt or solvate thereof.

8. A method according to claim 1, wherein the compound inhibits binding of leukotriene B4 to leukotriene B4 receptor by decreasing expression of leukotriene B4 receptor.

9. A method according to claim 8, wherein the compound is a nucleic acid molecule which binds to a nucleic acid molecule encoding leukotriene B4 receptor.

10. A method according to claim 1, wherein the compound inhibits binding of leukotriene B4 to leukotriene B4 receptor by binding leukotriene B4.

11. A method according to claim 1, wherein the compound inhibits binding of leukotriene B4 to leukotriene B4 receptor by inhibiting the production of leukotriene B4.

12. A method according to claim 11, wherein the compound inhibits the production of leukotriene B4 by inhibiting the conversion of leukotriene A4 to leukotriene B4.

13. A method according to claim 12, wherein the compound inhibits the conversion of leukotriene A4 to leukotriene B4 by binding to leukotriene A4.

14. A method according to claim 12, wherein the compound inhibits the conversion of leukotriene A4 to leukotriene B4 by inhibiting the activity of leukotriene A4 hydrolase.

15. A method according to claim 14, wherein the compound inhibits the activity of leukotriene A4 hydrolase by binding to leukotriene A4 hydrolase.

16. A method according to claim 14, wherein the compound inhibits the activity of leukotriene A4 hydrolase by decreasing expression of leukotriene A4 hydrolase.

17. A method according to claim 16, wherein the compound is a nucleic acid molecule which binds to a nucleic acid molecule encoding leukotriene A4 hydrolase.

18. A method according to claim 1, wherein the compound does not inhibit the production of leukotriene A4.

19. A method according to claim 1, wherein the compound is not a 5-lipoxygenase inhibitor.

20. A method according to claim 1, wherein the compound is not an inhibitor of 5-lipoxygenase-activating protein.

21. A method according to claim 1, wherein the compound is not a nucleic acid molecule which decreases expression of 5-lipoxygenase.

22. A method according to claim 1, wherein the adenocarcinoma cancer cells are present in a human subject and wherein said contacting comprises administering a therapeutically effective amount of the compound to the human subject.

23. A method of treating adenocarcinoma in a subject, said method comprising:
administering to the subject an amount of a compound effective to inhibit binding of leukotriene B4 to leukotriene B4 receptor.

24. A method according to claim 23, wherein the subject is a human subject.

25. A method according to claim 23, wherein the subject does not suffer from oral squamous cell carcinoma.

26. A method according to claim 23, wherein the amount is effective to decrease proliferation of cancer cells in the subject.

27. A method according to claim 23, wherein the amount is effective to induce apoptosis of cancer cells in the subject.

28. A method according to claim 23, wherein the amount is effective to induce differentiation of cancer cells in the subject into non-cancerous cells.

29. A method according to claim 23, wherein the compound inhibits binding of leukotriene B4 to leukotriene B4 receptor by binding to leukotriene B4 receptor.

30. A method according to claim 29, wherein the compound has the formula: 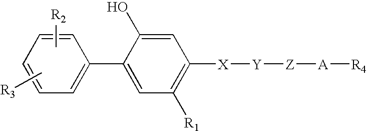
or a pharmaceutically acceptable salt or solvate thereof, wherein:
R₁ is C₁-C₅ alkyl, C₂-C₅ alkenyl, C₂-C₅ alkynyl, C₁-C₄ alkoxy, (C₁-C₄ alkyl)thio, halo, or R₂-substitutedphenyl;
each of R₂ and R₃ is independently hydrogen, halo, hydroxy, C₁-C₄ alkyl, C₁-C₄ alkoxy, (C₁-C₄ alkyl)-S(O)_{q}—, trifluoromethyl, or di-(C₁-C₃ alkyl)amino;
X is —O—, —S—, —C(═O), or —CH₂—, and Y is —O— or —CH₂—; or, when taken together, —X-Y— is —CH═CH— or —C≡C—;
Z is a straight or branched chain C₁-C₁₀ alkylidenyl;
A is a bond, —O—, —S—, —CH═CH—, or —CRₐR_{b}—, where each of Rₐ and R_{b} is independently hydrogen, C₁-C₅ alkyl, or R₇-substituted phenyl, or Rₐ and R_{b}, when taken together with the carbon atom to which they are attached, form a C₄-C₈ cycloalkyl ring;
R₄ is a R₆ or R₄ is a moiety having one of the following formulae: 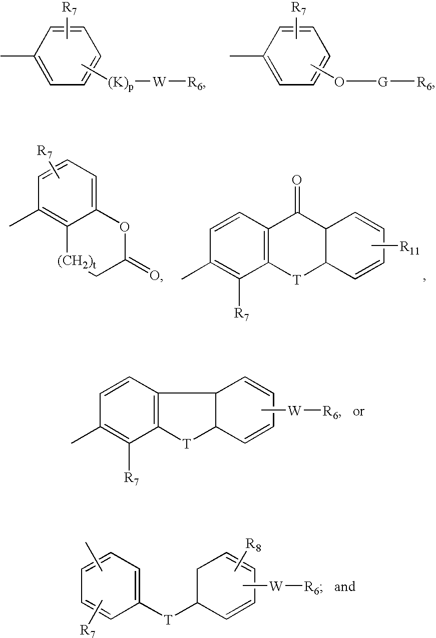
wherein:
each R₆ is independently —COOH, 5-tetrazolyl, —CON(R₉)₂, or —CONHSO₂R₁₀;
each R₇ is hydrogen, C₁-C₄ alkyl, C₂-C₅ alkenyl, C₂-C₅ alkynyl, benzyl, methoxy, —W—R₆, -T-G-R₆, (C₁-C₄ alkyl)-T-(C₃-C₄ alkylidenyl)-O—, or hydroxy;
R₈ is hydrogen or halo;
each R₉ is independently hydrogen, phenyl, or C₁-C₄ alkyl, or R₉, when taken together with the nitrogen atom to which they are attached, form a morpholino, piperidino, piperazino, or pyrrolidino group;
R₁₀ is C₁-C₄ alkyl or phenyl;
R₁₁ is R₂, —W—R₆, or -T-G-R₆;
each W is a bond or straight or branched chain divalent hydrocarbyl radical of one to eight carbon atoms;
each G is a straight or branched chain divalent hydrocarbyl radical of one to eight carbon atoms;
each T is a bond, —CH₂—, —O—, —NH—, —NHCO—, —C(═O)—, or —S(O)_{q}—;
K is —C(═O)— or —CH(OH)—;
each q is independently 0, 1, or 2;
p is 0 or 1; and
t is 0 or 1;
provided that, when X is —O— or —S—, Y is not —O—;
further provided that, when A is —O— or —S—, R₄ is not R₆;
further provided that, when A is —O— or —S— and Z is a bond, Y is not —O—; and
further provided that W is not a bond when p is 0.

31. A method according to claim 29, wherein the compound is selected from the group consisting of 2-(2-propyl-3-(3-(2-ethyl-4-(4-fluorophenyl)-5-hydroxyphenoxy)propoxy)phenoxy)benzoic acid, 3-(2-(3-(2-ethyl-4-(4-fluorophenyl)-5-hydroxyphenoxy)propoxy)-6-(4-carboxy-phenoxy)phenyl)propionic acid, 1-(4-(carboxy-methoxy)phenyl)-1-(1H-tetrazol-5-yl)-6-(2-ethyl-4-(4-fluorophenyl)-5-hydroxyphenoxy)hexane, 3-(4-(7-carboxy-9-oxo-3-(3-(2-ethyl-4-(4-fluorophenyl)-5-hydroxyphenoxy)-propoxy)-9H-xanthene))propanoic acid, 5-(3-(2-(1-carboxy)-ethyl)-4-(3-(2-ethyl-4-(4-fluorophenyl)-5-hydroxyphenoxy)-propoxy)phenyl)-4-pentynoic acid, a pharmaceutically acceptable salt or solvate thereof, and combinations thereof.

32. A method according to claim 29, wherein the compound is 2-(2-propyl-3-(3-(2-ethyl-4-(4-fluorophenyl)-5-hydroxyphenoxy)propoxy)phenoxy)benzoic acid or a pharmaceutically acceptable salt or solvate thereof.

33. A method according to claim 23, wherein the compound does not inhibit the production of leukotriene A4.

34. A method according to claim 23, wherein the adenocarcinoma is selected from the group consisting of prostate cancer, lung cancer, stomach cancer, breast cancer, colon cancer, pancreatic cancer, and combinations thereof.

35. A method of decreasing proliferation of adenocarcinoma cancer cells, or of inducing apoptosis of adenocarcinoma cancer cells, or of inducing differentiation of adenocarcinoma cancer cells into non-cancerous cells, said method comprising:
contacting a sample comprising adenocarcinoma cancer cells with a compound having the formula: 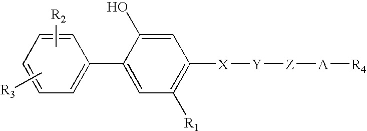
or a pharmaceutically acceptable salt or solvate thereof, wherein:
R₁ is C₁-C₅ alkyl, C₂-C₅ alkenyl, C₂-C₅ alkynyl, C₁-C₄ alkoxy, (C₁-C₄ alkyl)thio, halo, or R₂-substitutedphenyl;
each of R₂ and R₃ is independently hydrogen, halo, hydroxy, C₁-C₄ alkyl, C₁-C₄ alkoxy, (C₁-C₄ alkyl)-S(O)_{q}—, trifluoromethyl, or di-(C₁-C₃ alkyl)amino;
X is —O—, —S—, —C(═O), or —CH₂—, and Y is —O— or —CH₂—; or, when taken together, —X—Y— is —CH═CH— or —C≡C—;
Z is a straight or branched chain C₁-C₁₀ alkylidenyl;
A is a bond, —O—, —S—, —CH═CH—, or —CRₐR_{b}—, where each of Rₐ and R_{b} is independently hydrogen, C₁-C₅ alkyl, or R₇-substituted phenyl, or Rₐ and R_{b}, when taken together with the carbon atom to which they are attached, form a C₄-C₈ cycloalkyl ring;
R₄ is a R₆ or R₄ is a moiety having one of the following formulae: 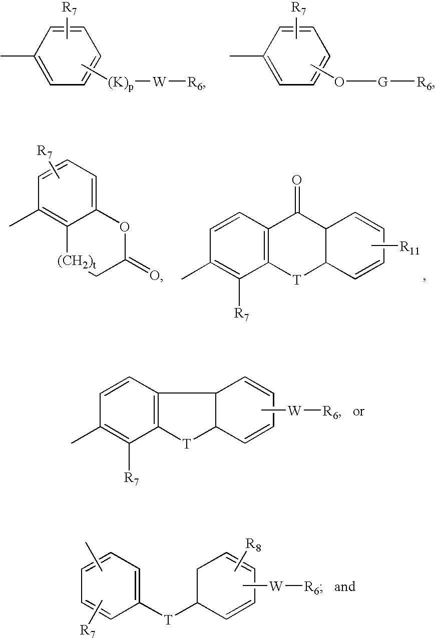
wherein:
each R₆ is independently —COOH, 5-tetrazolyl, —CON(R₉)₂, or —CONHSO₂R₁₀;
each R₇ is hydrogen, C₁-C₄ alkyl, C₂-C₅ alkenyl, C₂-C₅ alkynyl, benzyl, methoxy, —W—R₆, -T-G-R₆, (C₂-C₄ alkyl)-T-(C₁-C₄ alkylidenyl)-O—, or hydroxy;
R₈ is hydrogen or halo;
each R₉ is independently hydrogen, phenyl, or C₁-C₄ alkyl, or R₉, when taken together with the nitrogen atom to which they are attached, form a morpholino, piperidino, piperazino, or pyrrolidino group;
R₁₀ is C₁-C₄ alkyl or phenyl;
R₁₁ is R₂, —W—R₆, or -T-G-R₆;
each W is a bond or straight or branched chain divalent hydrocarbyl radical of one to eight carbon atoms;
each G is a straight or branched chain divalent hydrocarbyl radical of one to eight carbon atoms;
each T is a bond, —CH₂—, —O—, —NH—, —NHCO—, —C(═O)—, or —S(O)_{q}—;
K is —C(═O)— or —CH(OH)—;
each q is independently 0, 1, or 2;
p is 0 or 1; and
t is 0 or 1;
provided that, when X is —O— or —S—, Y is not —O—;
further provided that, when A is —O— or —S—, R₄ is not R₆;
further provided that, when A is —O— or —S— and Z is a bond, Y is not —O—; and
further provided that W is not a bond when p is 0.

36. A method according to claim 35, wherein the compound is selected from the group consisting of 2-(2-propyl-3-(3-(2-ethyl-4-(4-fluorophenyl)-5-hydroxyphenoxy)propoxy)phenoxy)benzoic acid, 3-(2-(3-(2-ethyl-4-(4-fluorophenyl)-5-hydroxyphenoxy)propoxy)-6-(4-carboxy-phenoxy)phenyl)propionic acid, 1-(4-(carboxy-methoxy)phenyl)-1-(1H-tetrazol-5-yl)-6-(2-ethyl-4-(4-fluorophenyl)-5-hydroxyphenoxy)hexane, 3-(4-(7-carboxy-9-oxo-3-(3-(2-ethyl-4-(4-fluorophenyl)-5-hydroxyphenoxy)-propoxy)-9H-xanthene))propanoic acid, 5-(3-(2-(1-carboxy)-ethyl)-4-(3-(2-ethyl-4-(4-fluorophenyl)-5-hydroxyphenoxy)-propoxy)phenyl)-4-pentynoic acid, a pharmaceutically acceptable salt or solvate thereof, and combinations thereof.

37. A method according to claim 35, wherein the compound is 2-(2-propyl-3-(3-(2-ethyl-4-(4-fluorophenyl)-5-hydroxyphenoxy)propoxy)phenoxy)benzoic acid or a pharmaceutically acceptable salt or solvate thereof.

38. A method according to claim 35, wherein the sample does not comprise oral squamous cell carcinoma cells.

39. A method according to claim 35, wherein the sample comprises prostate cancer cells, lung cancer cells, stomach cancer cells breast cancer cells, pancreatic cancer cells, colon cancer cells, or combinations thereof.

40. A method of treating adenocarcinoma in a subject, said method comprising:
administering to the subject a therapeutically effective amount of a compound having the formula: 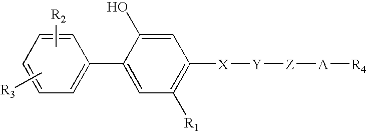
or a pharmaceutically acceptable salt or solvate thereof, wherein:
R₁ is C₁-C₅ alkyl, C₂-C₅ alkenyl, C₂-C₅ alkynyl, C₁-C₄ alkoxy, (C₁-C₄ alkyl)thio, halo, or R₂-substitutedphenyl;
each of R₂ and R₃ is independently hydrogen, halo, hydroxy, C₁-C₄ alkyl, C₁-C₄ alkoxy, (C₁-C₄ alkyl)-S(O)_{q}—, trifluoromethyl, or di-(C₁-C₃ alkyl)amino;
X is —O—, —S—, —C(═O), or —CH₂—, and Y is —O— or —CH₂—; or, when taken together, —X-Y— is —CH═CH— or —C≡C—;
Z is a straight or branched chain C₁-C₁₀ alkylidenyl;
A is a bond, —O—, —S—, —CH═CH—, or —CRₐR_{b}—, where each of Rₐ and R_{b} is independently hydrogen, C₁-C₅ alkyl, or R₇-substituted phenyl, or Rₐ and R_{b}, when taken together with the carbon atom to which they are attached, form a C₄-C₈ cycloalkyl ring;
R₄ is a R₆ or R₄ is a moiety having one of the following formulae: 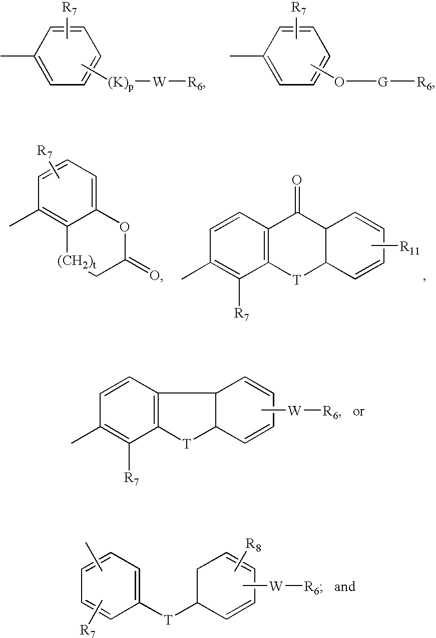
wherein:
each R₆ is independently —COOH, 5-tetrazolyl, —CON(R₉)₂, or —CONHSO₂R₁₀;
each R₇ is hydrogen, C₁-C₄ alkyl, C₂-C₅ alkenyl, C₂-C₅ alkynyl, benzyl, methoxy, —W—R₆, -T-G-R₆, (C₁-C₄ alkyl)-T-(C₁-C₄ alkylidenyl)-O—, or hydroxy;
R₈ is hydrogen or halo;
each R₉ is independently hydrogen, phenyl, or C₁-C₄ alkyl, or R₉, when taken together with the nitrogen atom to which they are attached, form a morpholino, piperidino, piperazino, or pyrrolidino group;
R₁₀ is C₁-C₄ alkyl or phenyl;
R₁₁ is R₂, —W—R₆, or -T-G-R₆;
each W is a bond or straight or branched chain divalent hydrocarbyl radical of one to eight carbon atoms;
each G is a straight or branched chain divalent hydrocarbyl radical of one to eight carbon atoms;
each T is a bond, —CH₂—, —O—, —NH—, —NHCO—, —C(═O)—, or —S(O)_{q}—;
K is —C(═O)— or —CH(OH)—;
each q is independently 0, 1, or 2;
p is 0 or 1; and
t is 0 or 1;
provided that, when X is —O— or —S—, Y is not —O—;
further provided that, when A is —O— or —S—, R₄ is not R₆;
further provided that, when A is —O— or —S— and Z is a bond, Y is not —O—; and
further provided that W is not a bond when p is 0.

41. A method according to claim 40, wherein the compound is selected from the group consisting of 2-(2-propyl-3-(3-(2-ethyl-4-(4-fluorophenyl)-5-hydroxyphenoxy)propoxy)phenoxy)benzoic acid, 3-(2-(3-(2-ethyl-4-(4-fluorophenyl)-5-hydroxyphenoxy)propoxy)-6-(4-carboxy-phenoxy)phenyl)propionic acid, 1-(4-(carboxy-methoxy)phenyl)-1-(1H-tetrazol-5-yl)-6-(2-ethyl-4-(4-fluorophenyl)-5-hydroxyphenoxy)hexane, 3-(4-(7-carboxy-9-oxo-3-(3-(2-ethyl-4-(4-fluorophenyl)-5-hydroxyphenoxy)-propoxy)-9H-xanthene))propanoic acid, 5-(3-(2-(1-carboxy)-ethyl)-4-(3-(2-ethyl-4-(4-fluorophenyl)-5-hydroxyphenoxy)-propoxy)phenyl)-4-pentynoic acid, a pharmaceutically acceptable salt or solvate thereof, and combinations thereof.

42. A method according to claim 40, wherein the compound is 2-(2-propyl-3-(3-(2-ethyl-4-(4-fluorophenyl)-5-hydroxyphenoxy)propoxy)phenoxy)benzoic acid or a pharmaceutically acceptable salt or solvate thereof.

43. A method according to claim 40, wherein the subject is a human subject.

44. A method according to claim 40, wherein the subject does not suffer from oral squamous cell carcinoma.

45. A method according to claim 40, wherein the adenocarcinoma is selected from the group consisting of prostate cancer, lung cancer, stomach cancer, breast cancer, colon cancer, pancreatic cancer, and combinations thereof.
